(19) United States
(12) Patent Application Publication
(10) Pub. No.: US 2018/0084901 A1
(43) Pub. Date: Mar. 29, 2018

(54) TOOTHBRUSH HOLDER WITH BRISTLE TREATMENT LIQUID DISPENSER

(71) Applicant: MAXOR INC., DRUMMONDVILLE (CA)

(72) Inventors: DENIS LAMOTHE, DRUMMONDVILLE (CA); JEAN LAMOTHE, DRUMMONDVILLE (CA)

(21) Appl. No.: 15/562,640

(22) PCT Filed: Apr. 11, 2016
(86) PCT No.: PCT/CA2016/050411

§ 371 Date: Sep. 28, 2017

Related U.S. Application Data
(60) Provisional application No. 62/146,292, filed on Apr. 11, 2015.

(30) Foreign Application Priority Data
Apr. 13, 2015 (CA) 2887522

(51) Int. Cl. A46B 17/06; A46B 17/04; A61L 2/22; A47K 1/09

(52) U.S. Cl. CPC A46B 17/065; A46B 17/04; A61L 2202/15; A47K 1/09; A46B 2200/1066; A61L 2/22

(57) ABSTRACT

A toothbrush holder enclosure is provided which includes: a back plate, a resilient clamp, a front cover and a sanitation agent dispenser. The back plate and front cover form a housing. The clamp is made of a resilient material giving it a springy property and shaped to urge two clamp arms towards one another in order for the clamp to grip a toothbrush. Two rollers at the tips of the clamp arms engage a toothbrush head inserted in the clamp as the clamp is spread apart from the clamped position to a deviated state. With the toothbrush inserted, the rollers exert pressure on a neck of the toothbrush to hold the toothbrush fast in place with bristles generally oriented and generally aligned with a dispensing orifice supplied with sanitation agent from a reservoir of the dispenser. An actuator causes sanitation agent to issue from the dispensing orifice.

### TECHNICAL FIELD

This invention relates to toothbrush storage and sanitization, and in particular to a tidy toothbrush holder apparatus for storage and sanitation of a toothbrush.

### BACKGROUND

There is a need for providing sanitation with respect to stored toothbrushes.

Applying a sterilizing liquid to toothbrushes is known in the art. In some cases, the toothbrush was stored upside down in a bath of sterile solution. In other cases, the toothbrush was presentable to a station where a solution could be sprayed onto the head of the toothbrush.

One type of toothbrush enclosure provides tidiness by affixing a toothbrush enclosure to a wall. International Published Pat. Appl. No. WO 1998/035,585 (PUURUNEN) describes a toothbrush rack generally having three parts: back plate for affixing the rack to a wall, a holder for engaging a toothbrush head from the sides and a shell to provide an enclosure. The toothbrush is secured in the rack by three point retention between fingers of the holder and prevented from rotating by resting on the back plate. Unfortunately such racks are less sanitary than desired because the back plate which touches the toothbrush cannot be readily removed for cleaning. Also more modern toothbrush designs incorporating soft materials tend to get lodged in, which requires disassembly of the rack for removal of the toothbrush.

A number of attempts have been proposed for storing conventional toothbrushes in sanitation solution, which are cumbersome and unsuitable for travel. Some attempts have been proposed at spraying a toothbrush with a sanitizing agent:

US Published Pat. Appl. No. 2002/0031461 (KNIPP) discloses a container for holding several toothbrushes in a common chamber into which an antibacterial toothbrush spray is dispensed. A pump mechanism is actuated by depressing a lid. No enabling disclosure is provided for drawing antibacterial/antiviral sanitizing liquid from an internal reservoir located in a middle central column of the container and for the spraying of the sanitizing liquid from small openings aligned with brush ends of toothbrushes. Unfortunately, communal storage of multiple toothbrushes increases the chances of cross-contamination between the toothbrushes leading to re-introduction of lingering infective agents.

U.S. Pat. No. 6,253,773 (INGEMANN) discloses a portable dental hygiene kit with a treatment space and a spray device which sprays a treatment medium into the treatment space. The spray device and the parts to be sprayed with the treatment medium are separated from one another but arranged in spray relation to one another on a holder which encloses the spray device and the parts. A spray head of the spray device has a lateral nozzle. A cap is provided as a trigger element for spray mist to be released. When the detachable cap is placed on the holding device in one of the possible placement positions, it secures the spray device against release of a spray stream, and in the other possible placement position, it can cause a spray stream to be released. Unfortunately, when removed as designed, the cap can be easily dropped and contaminated. Loss of the cap renders the kit ineffective to disinfect.

International Published Pat. Appl. No. WO 1996/016,750 (HECKER et al.) discloses a dental care station where the head part of a toothbrush and the head of a spray can are in spraying relation to each other. A two-part case-like holding device is provided. The spray can sits in a receptacle cavity of one part of the holding device, and the toothbrush is in the other part of the holding device. Guide rails position the toothbrush when the device is closed. The spray head of the valve of the spray can, which is designed as a lateral valve, sits in a brush chamber. A spray mist is released by means of a release button which is provided on the head of the housing and can act on the bottom of the spray can. By depressing the release button, the spray mist can be directed at the bristles of the toothbrush in the brush chamber in a controlled manner. Unfortunately, a spray stream may be released unintentionally by depressing the release button inadvertently. This danger is especially great in luggage in particular.

U.S. Pat. No. 6,099,813 (GIPSON) discloses a unitary toothbrush case receptacle having a bristle chamber and a handle chamber separated by a fluid seal. The case has ports with unidirectional flow valves through which sterilizing agents in the form of aerosol spray or fluid may be selectively admitted into the brush compartment or the handle compartment. This toothbrush case is not suitable for travel as it would be cumbersome to carry a separate aerosol canister.

### SUMMARY

Applicant has found that storage of toothbrushes by providing individual toothbrush head enclosures is preferred over communal toothbrush enclosures, and that storage of toothbrushes without any sanitizing agent being involved is perceived as being less sanitary than the use of a sanitization agent. Applicant proposes herein the combination of individual toothbrush storage using a toothbrush head enclosure with an application device to apply a liquid sanitization agent to the bristles of the toothbrush. In this way, the liquid sanitization agent application is associated with the individual storage enclosure so that the need for a toothbrush head enclosure and the need for sanitization are satisfied. Such association can be done by configuring the application device to apply the sanitization agent onto bristles of the toothbrush head while secured in the enclosure. Such association can also be done by configuring the application device to apply the sanitization agent onto bristles of the toothbrush head while removed from the enclosure.

Unsuitable prior art devices generally relate to toothbrush storage enclosure which are limited to providing a basin for dipping a toothbrush into a solution (mouthwash, disinfectant, etc.). These devices are complicated to use as they are large and they are unsuited for travel as the solution in the basin can spill while in transit. Some prior art devices relate to spraying a toothbrush with a sanitizing agent but unsuitably lack protection against dispensing sanitizing agent accidentally. Most of these prior art devices provide a relatively large communal/familial station for holding multiple toothbrushes typically loosely during treatment and are not suitable as an individual brush enclosure for travel. Other prior art enclosures for individual toothbrushes lack compactness unsuitably being more concerned with various kits and cases undesirably increasing the required travel volume.

To address at least some of these concerns a proposed way to hold a toothbrush includes receiving the toothbrush head into a holder enclosure for example by gripping the toothbrush head by the sides of the head. The enclosure includes a reservoir of sanitizing agent (liquid, gel, paste, etc.). The enclosure in configured to protect the toothbrush head while allowing dispensing a small amount of sanitizing agent onto the bristles.

Some of shortcomings in the prior art toothbrush holders are addressed by employing a toothbrush holder apparatus described in International Published Pat. Appl. WO 2014/078953 (MAUFETTE et al.), which is incorporated herein by reference. The toothbrush holder apparatus includes as functional parts: an enclosure housing a resilient clamp. The housing includes back plate and front cover providing the toothbrush enclosure. The clamp part has a general inverted “U” shape and is preferably made of a resilient material giving it a springy property. The clamp is shaped to urge two gripping arms towards one another in order for the overall clamp to act as retainer. Preferably two rollers at the tips of the gripping arms engage a toothbrush head inserted in the clamp as the clamp is spread apart from a clamped position to a deviated state. With the toothbrush inserted, the rollers exert pressure on the neck of the toothbrush to hold the toothbrush fast in place. The rolling engagement action of the rollers provides retention in the toothbrush enclosure of toothbrushes having heads fitted with rubber sides and enables a much more fluid motion of the toothbrush with respect to the toothbrush holder. Small tabs can be integrated into the clamp, which in combination with the back plate, limit the toothbrush to a single orientation as it is inserted into the enclosure.

In accordance with the proposed solution a sanitary transport toothbrush holder apparatus is provided including as functional parts: an enclosure housing a resilient clamp and a reservoir of sanitation agent. The toothbrush holder is configured to retain a toothbrush head in opposing relation to a dispensing orifice through which sanitation agent is controllably dispensed towards toothbrush head. In some embodiments, the apparatus is configured to dispense sanitation agent by spray action in which case the orifice can include a nozzle. Preferably the sanitation agent can be sprayed onto the toothbrush bristles. In other embodiments, the sanitation agent is dispensed through the orifice as a globule of gel or paste forming a meniscus. The bristles of the toothbrush when retained in the clamp are positioned to wick up sanitation agent for example by capillary action.

In accordance with the proposed solution, the toothbrush holder includes release means for causing controlled dispensation of sanitation agent. In some implementations, the release means includes a release trigger, for example including a button causing the reservoir to dispense a measured amount of sanitation agent. In other implementations, the release means includes deforming the enclosure to cause the reservoir to dispense a measured amount of sanitation agent. In further implementations, the release means includes deforming the reservoir to dispense a measured amount of sanitation agent. In further implementations, the release means includes deforming the clamp to cause the reservoir to dispense a measured amount of sanitation agent. In yet other implementations, the release means includes ratcheting means configured to cause the reservoir to deform to dispense a measured amount of sanitation agent. In some implementations, the ratcheting means include a ratchet actuated by the insertion of the toothbrush head into the toothbrush holder. In other implementations, the ratcheting means include at least a roller having a ratcheting gear actuated by the insertion of the toothbrush head into the toothbrush holder.

In accordance with some embodiments of the proposed solution, there is provided a toothbrush holder for holding an individual toothbrush, the holder comprising: a toothbrush head enclosure for providing protection against bristle contact with foreign objects, the enclosure being dimensioned for receiving a head of a toothbrush while leaving a handle of the toothbrush exposed; a clip mechanism contained within the enclosure for securely receiving said head and allowing for release of said head; and a dispenser connected with said enclosure for dispensing sanitizing agent onto bristles of said head.

In accordance with some embodiments of the proposed solution, there is provided a kit comprising a toothbrush head holder comprising a toothbrush head enclosure for providing protection against bristle contact with foreign objects, the enclosure being dimensioned for receiving a head of a toothbrush while leaving a handle of the toothbrush exposed, and a dispenser for dispensing sanitizing agent onto bristles of the toothbrush head. Thus, it is contemplated that the dispenser be a separate component to the enclosure or integrated, while providing the dual function of dispensing onto bristles a suitable agent and protecting a toothbrush head using the holder.

Without limiting the invention, the above mentioned engagement features for retaining a toothbrush in a toothbrush holder apparatus, in accordance with some embodiments of the proposed solution, provide synergistic advantages derived for example from an improved retention between a toothbrush holder clip having rollers, a toothbrush having rubberized toothbrush head sides and sanitation.

### BRIEF DESCRIPTION OF THE DRAWINGS

The proposed solution can be better understood by way of the following detailed description of embodiments of the invention with reference to the appended drawings, in which:

FIG. 1A is a schematic diagram illustrating a top view of a toothbrush holder enclosure in accordance with a first embodiment of the proposed solution;

FIG. 1B is a schematic diagram illustrating a front view of a toothbrush holder enclosure in accordance with the first embodiment of the proposed solution;

FIG. 1C is a schematic diagram illustrating a right side view of a toothbrush holder enclosure in accordance with the first embodiment of the proposed solution;

FIG. 1D is a schematic diagram illustrating a perspective view of a toothbrush holder enclosure in accordance with the first embodiment of the proposed solution;

FIG. 2A is a schematic diagram illustrating a top view of a back plate in accordance with a first implementation of the first embodiment of the toothbrush holder enclosure of the proposed solution;

FIG. 2B is a schematic diagram illustrating a front view of the back plate in accordance with the first implementation of the first embodiment of the toothbrush holder enclosure of the proposed solution;

FIG. 2C is a schematic diagram illustrating a right side view of the back plate in accordance with the first implementation of the first embodiment of the toothbrush holder enclosure of the proposed solution;

FIG. 2D is a schematic diagram illustrating a back view of the back plate in accordance with the first implementation of the first embodiment of the toothbrush 15 holder enclosure of the proposed solution;

FIG. 2E is a schematic diagram illustrating a perspective view of the back plate in accordance with the first implementation of the first embodiment of the toothbrush holder enclosure of the proposed solution;

FIG. 3A is a schematic diagram illustrating a front view of another back plate in accordance with a second implementation of the first embodiment of the toothbrush holder enclosure of the proposed solution;

FIG. 3B is a schematic diagram illustrating a bottom view of the back plate in accordance with the second implementation of the first embodiment of the toothbrush holder enclosure of the proposed solution;

FIG. 3C is a schematic diagram illustrating a bottom perspective view of the back plate in accordance with the second implementation of the first embodiment of the toothbrush holder enclosure of the proposed solution;

FIG. 3D is a schematic diagram illustrating a top perspective view of the back plate in accordance with the second implementation of the first embodiment of the toothbrush holder enclosure of the proposed solution;

FIG. 4A is a schematic diagram illustrating a front view of a back plate and toothbrush grip element combination in accordance with the second implementation of the first embodiment of the toothbrush holder of the proposed solution;

FIG. 4B is a schematic diagram illustrating a bottom view of the back plate and toothbrush grip element combination in accordance with the second implementation of the first embodiment of the toothbrush holder of the proposed solution;

FIG. 4C is a schematic diagram illustrating a right side view of the back plate and toothbrush grip element combination in accordance with the second implementation of the first embodiment of the toothbrush holder of the proposed solution;

FIG. 4D is a schematic diagram illustrating a bottom perspective view of the back plate and toothbrush grip element combination in accordance with the second implementation of the first embodiment of the toothbrush holder of the proposed solution;

FIG. 4E is a schematic diagram illustrating a top perspective view of the back plate and toothbrush grip element combination in accordance with the second implementation of the first embodiment of the toothbrush holder of the proposed solution;

FIG. 5A is a schematic diagram illustrating a top view of a toothbrush grip element in accordance with a first implementation of the proposed solution;

FIG. 5B is a schematic diagram illustrating a front view of the toothbrush grip element in accordance with the first implementation of the proposed solution;

FIG. 5C is a schematic diagram illustrating a bottom view of the toothbrush grip element in accordance with the first implementation of the proposed solution;

FIG. 5D is a schematic diagram illustrating a right side view of the toothbrush grip element in accordance with the first implementation of the proposed solution;

FIG. 5E is a schematic diagram illustrating a back view of the toothbrush grip element in accordance with the first implementation of the proposed solution;

FIG. 5F is a schematic diagram illustrating a bottom perspective view of the toothbrush grip element in accordance with the first implementation of the proposed solution;

FIG. 5G is a schematic diagram illustrating a top perspective view of the clip toothbrush grip element in accordance with the first implementation of the proposed solution;

FIG. 6A is a schematic diagram illustrating a top view of a toothbrush grip element in accordance with a second implementation of the proposed solution;

FIG. 6B is a schematic diagram illustrating a front view of the toothbrush grip element in accordance with the second implementation of the proposed solution;

FIG. 6C is a schematic diagram illustrating a bottom view of the toothbrush grip element in accordance with the second implementation of the proposed solution;

FIG. 6D is a schematic diagram illustrating a right side view of the toothbrush grip element in accordance with the second implementation of the proposed solution;

FIG. 6E is a schematic diagram illustrating a top perspective view of the toothbrush grip element in accordance with the second implementation of the proposed solution;

FIG. 6F is a schematic diagram illustrating a bottom perspective view of the toothbrush grip element in accordance with the second implementation of the proposed solution;

FIG. 6G is a schematic diagram illustrating a another bottom perspective view of the toothbrush grip element in accordance with the second implementation of the proposed solution;

FIG. 7A is a schematic diagram illustrating a perspective see-through view of a sanitizing toothbrush holder in accordance with an implementation of the proposed solution;

FIG. 7B is a schematic diagram illustrating a perspective exploded view of a sanitizing toothbrush holder in accordance with an implementation of the proposed solution;

FIG. 7C is a schematic diagram illustrating a perspective view of an open sanitizing toothbrush holder for loading in a sanitation agent dispenser cartridge in accordance with another implementation of the proposed solution;

FIG. 7D is a schematic diagram illustrating an exploded view of a sanitizing toothbrush holder having a sanitation agent dispenser cartridge (in relation to a toothbrush) in accordance with implementation of the proposed solution illustrated in FIG. 7C;

FIG. 7E is a schematic diagram illustrating a perspective view of the sanitizing toothbrush holder without the front cover in accordance with the implementation of the proposed solution illustrated in FIG. 7C;

FIGS. 7F and 7G are schematic diagrams illustrating respectively a top and right side view of the sanitizing toothbrush holder without the front cover in accordance with the implementation of the proposed solution illustrated in FIGS. 7C, 7D and 7E;

FIG. 7H is a schematic diagram illustrating a top view of the assembled sanitizing toothbrush holder in accordance with the implementation of the proposed solution illustrated in FIGS. 7C, 7D, 7E, 7F and 7G;

FIGS. 7I and 7J are schematic diagrams illustrating right side views of the sanitizing toothbrush holder in operation between a normal position in FIG. 7I and an actuated position in FIG. 7J in accordance with the implementation of the proposed solution illustrated in FIGS. 7C, 7D, 7E, 7F, 7G and 7H;

FIG. 8A is a schematic diagram illustrating a top view of a front cover in accordance with the first embodiment of the toothbrush holder enclosure of the proposed solution;

FIG. 8B is a schematic diagram illustrating a front view of the front cover in accordance with the first embodiment of the toothbrush holder enclosure of the proposed solution;

FIG. 8C is a schematic diagram illustrating a right side view of the front cover in accordance with the first embodiment of the toothbrush holder enclosure of the proposed solution;

FIG. 8D is a schematic diagram illustrating a back view of the front cover in accordance with the first embodiment of the toothbrush holder enclosure of the proposed solution;

FIG. 9A is a schematic diagram illustrating a top view of a front cover window in accordance with the first embodiment of the toothbrush holder enclosure of the proposed solution;

FIG. 9B is a schematic diagram illustrating a front view of the front cover window in accordance with the first embodiment of the toothbrush holder enclosure of the proposed solution;

FIG. 9C is a schematic diagram illustrating a left side view of the front cover window in accordance with the first embodiment of the toothbrush holder enclosure of the proposed solution;

FIG. 9D is a schematic diagram illustrating a back view of the front cover window in accordance with the first embodiment of the toothbrush holder enclosure of the proposed solution;

FIG. 9E is a schematic diagram illustrating a perspective view of the front cover window in accordance with the first embodiment of the toothbrush holder enclosure of the proposed solution;

FIG. 10A is a schematic diagram illustrating a front view of another back plate in accordance with a fourth implementation of the first embodiment of the toothbrush holder enclosure of the proposed solution;

FIG. 10B is a schematic diagram illustrating a back view of the back plate in accordance with the fourth implementation of the first embodiment of the toothbrush holder enclosure of the proposed solution;

FIG. 10C is a schematic diagram illustrating a top view of the back plate in accordance with the fourth implementation of the first embodiment of the toothbrush holder enclosure of the proposed solution;

FIG. 10D is a schematic diagram illustrating a bottom view of the back plate in accordance with the fourth implementation of the first embodiment of the toothbrush holder enclosure of the proposed solution;

FIG. 10E is a schematic diagram illustrating a right side view of the back plate in accordance with the fourth implementation of the first embodiment of the toothbrush holder enclosure of the proposed solution;

FIG. 10F is a schematic diagram illustrating top perspective view of the back plate in accordance with the fourth implementation of the first embodiment of the toothbrush holder enclosure of the proposed solution;

FIG. 10G is a schematic diagram illustrating a bottom perspective view of the back plate in accordance with the fourth implementation of the first embodiment of the toothbrush holder enclosure of the proposed solution;

FIG. 11A is a schematic diagram illustrating a front view of a toothbrush grip element in accordance with the fourth implementation of the first embodiment of the proposed solution;

FIG. 11B is a schematic diagram illustrating a back view of the toothbrush grip element in accordance with the fourth implementation of the first embodiment of the proposed solution;

FIG. 11C is a schematic diagram illustrating a top view of the toothbrush grip element in accordance with the fourth implementation of the first embodiment of the proposed solution;

FIG. 11D is a schematic diagram illustrating a bottom view of the toothbrush grip element in accordance with the fourth implementation of the first embodiment of the proposed solution;

FIG. 11E is a schematic diagram illustrating a right side view of the toothbrush grip element in accordance with the fourth implementation of the first embodiment of the proposed solution;

FIG. 11F is a schematic diagram illustrating a bottom perspective view of the toothbrush grip element in accordance with the fourth implementation of the first embodiment of the proposed solution;

FIG. 11G is a schematic diagram illustrating a top perspective view of the clip toothbrush grip element in accordance with the fourth implementation of the first embodiment of the proposed solution;

FIG. 12A is a schematic diagram illustrating a front view of a back plate and toothbrush grip element combination in accordance with the fourth implementation of the first embodiment of the toothbrush holder of the proposed solution;

FIG. 12B is a schematic diagram illustrating a bottom view of the back plate and toothbrush grip element combination in accordance with the fourth implementation of the first embodiment of the toothbrush holder of the proposed solution;

FIG. 12C is a schematic diagram illustrating a right side view of the back plate and toothbrush grip element combination in accordance with the fourth implementation of the first embodiment of the toothbrush holder of the proposed solution;

FIG. 12D is a schematic diagram illustrating a top perspective view of the back plate and toothbrush grip element combination in accordance with the fourth implementation of the first embodiment of the toothbrush holder of the proposed solution;

FIG. 12E is a schematic diagram illustrating a bottom perspective view of the back plate and toothbrush grip element combination in accordance with the fourth implementation of the first embodiment of the toothbrush holder of the proposed solution;

FIG. 13A is a schematic diagram illustrating a front view of a front cover in accordance with the fourth implementation of the first embodiment of the toothbrush holder enclosure of the proposed solution;

FIG. 13B is a schematic diagram illustrating a back view of the front cover in accordance with the fourth implementation of the first embodiment of the toothbrush holder enclosure of the proposed solution;

FIG. 13C is a schematic diagram illustrating a top view of the front cover in accordance with the fourth implementation of the first embodiment of the toothbrush holder enclosure of the proposed solution;

FIG. 13D is a schematic diagram illustrating a right side view of the front cover in accordance with the fourth implementation of the first embodiment of the toothbrush holder enclosure of the proposed solution;

FIG. 13E is a schematic diagram illustrating a bottom perspective view of the front cover in accordance with the fourth implementation of the first embodiment of the 15 toothbrush holder enclosure of the proposed solution;

FIG. 13F is a schematic diagram illustrating a top perspective view of the front cover in accordance with the fourth implementation of the first embodiment of the toothbrush holder enclosure of the proposed solution;

FIG. 14A is a schematic diagram illustrating a front view of a front cover window in accordance with the fourth implementation of the first embodiment of the toothbrush holder enclosure of the proposed solution;

FIG. 14B is a schematic diagram illustrating a back view of the front cover window in accordance with the fourth implementation of the first embodiment of the toothbrush holder enclosure of the proposed solution;

FIG. 14C is a schematic diagram illustrating a top view of the front cover window in accordance with the fourth implementation of the first embodiment of the toothbrush holder enclosure of the proposed solution;

FIG. 14D is a schematic diagram illustrating a right side view of the front cover window in accordance with the fourth implementation of the first embodiment of the toothbrush holder enclosure of the proposed solution;

FIG. 14E is a schematic diagram illustrating a bottom perspective view of the front cover window in accordance with the fourth implementation of the first embodiment of the toothbrush holder enclosure of the proposed solution;

FIG. 14F is a schematic diagram illustrating a top perspective view of the front cover window in accordance with the fourth implementation of the first embodiment of the toothbrush holder enclosure of the proposed solution;

FIG. 15A shows a front view of a toothbrush holder having an enclosure for receiving a toothbrush head and having an integrated sanitization fluid dispenser for dispensing fluid onto bristles of the toothbrush while the toothbrush is removed from the enclosure;

FIG. 15B shows a side view corresponding to FIG. 15A;

FIG. 16 is a perspective exploded view of a sanitizing toothbrush holder in accordance with an implementation of the proposed solution;

FIG. 17 is a perspective view of the assembled sanitizing toothbrush holder in accordance with the implementation of the proposed solution illustrated in FIG. 16;

FIG. 18 is a perspective view of the back cover of the sanitizing toothbrush holder shown in FIG. 16;

FIG. 19 is a perspective view of a reservoir of the sanitizing toothbrush holder shown in FIG. 16;

FIG. 20 is a side sectional view of the reservoir shown in FIG. 19,

wherein similar features bear similar labels throughout the drawings. References to position qualifiers such as, but not limited to “top” and “bottom”, in the present specification is made solely with reference to the orientation of the drawings as presented in the application and do not imply any absolute spatial orientation.

### DETAILED DESCRIPTION

When a factory-fresh toothbrush is first used, it provides a sense of comfort that it is clean and safe to use. Once it is used, it has come in contact with oral bacteria and any other germs present in the oral cavity. Rinsing the toothbrush gives some sense of hygiene, however, there is a lingering sense that bristles of an exposed toothbrush head may contain remnant bacteria or germs from the oral cavity and may attract airborne germs as well.

Giving the user a feeling of comfort and security in storage of the toothbrush involves (1) providing a way for the toothbrush to be stored without exposure of the bristles to airborne contaminants and with protection of the bristles against physical contact with other objects, and (2) sanitizing the bristles with the application of a sanitization agent to guard against the impression that any remaining or newly arrived germs could be present and multiplying on the surfaces of the bristles. The combination of these two elements is sufficiently reassuring to most users that the toothbrush is as good as a factory-fresh toothbrush each time the toothbrush is used. The user can be further comforted by an awareness of the use of the sanitization agent by a flavor imparted to the bristles from the sanitization agent. While such a flavor is added to any flavor of the toothpaste, it can be made to be noticed. For example, a flavor associated with many mouthwash solutions can be distinctive from a toothpaste flavor. A natural, antimicrobial oil, such as an essential oil like thyme, clove, lemon, oregano, basil, cinnamon, rosemary, and the like, would have a pleasant flavor distinct from a typical toothpaste flavor, such as mint.

This can be achieved with a minimum of infrastructure and can be quite compact. Bristles can be protected by an enclosure against airborne contaminants. The toothbrush handle need not be protected as the typically smooth surfaces attract fewer contaminants than the bristles. In some embodiments described below, concavely shaped rollers are used to safely guide the head and neck of the toothbrush body into the enclosure without exposing the bristles to contact with the enclosure due to a twisting or misalignment motion. In these cases, the holder is compatible with most commonly commercial toothbrush heads. Of course, a toothbrush head can be adapted for use with a specific holder design to provide for storage with limited risk of the bristles making physical contact with the holder as the head is inserted and removed.

For the combination of these two elements to be effective, an applicator of a sanitization agent can be used to apply liquid to the bristles even on the outside of the enclosure, preferably as long as the enclosure is able to receive and discharge the toothbrush head with little chance of physical contact with the bristles. Alternatively, the applicator can be internal to the enclosure such that sanitization liquid can be applied after insertion for storage.

### Toothbrush Holder

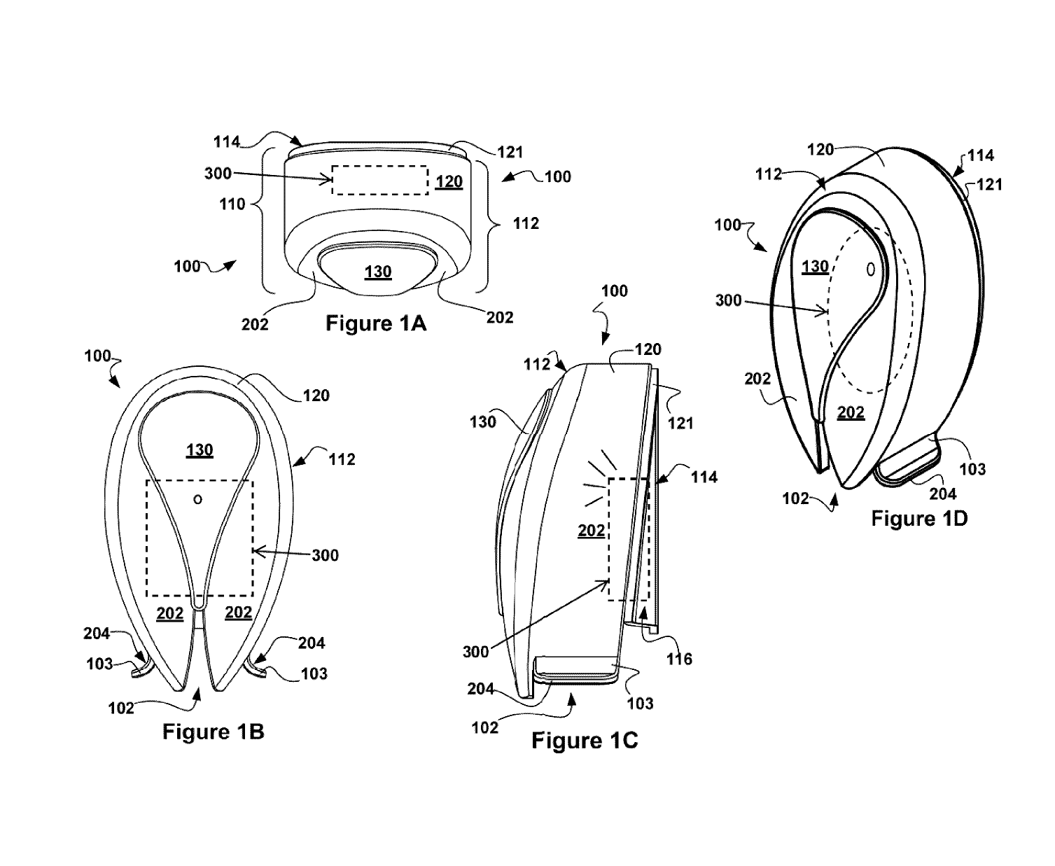

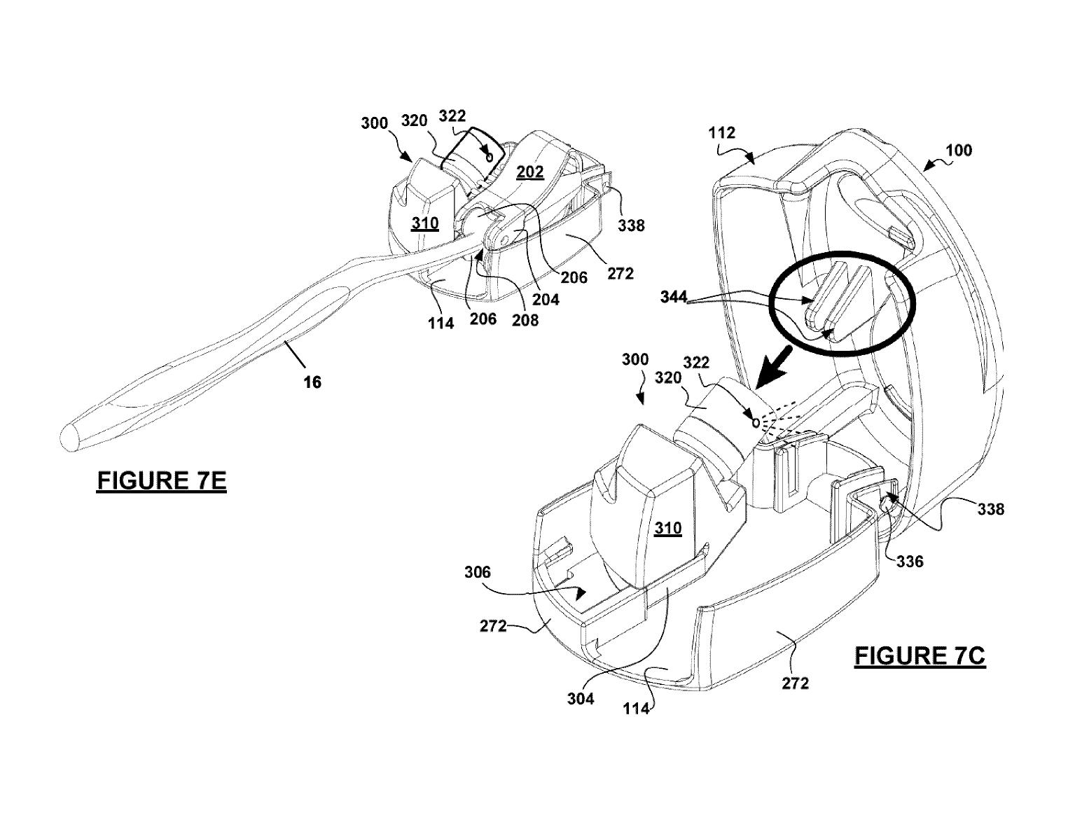

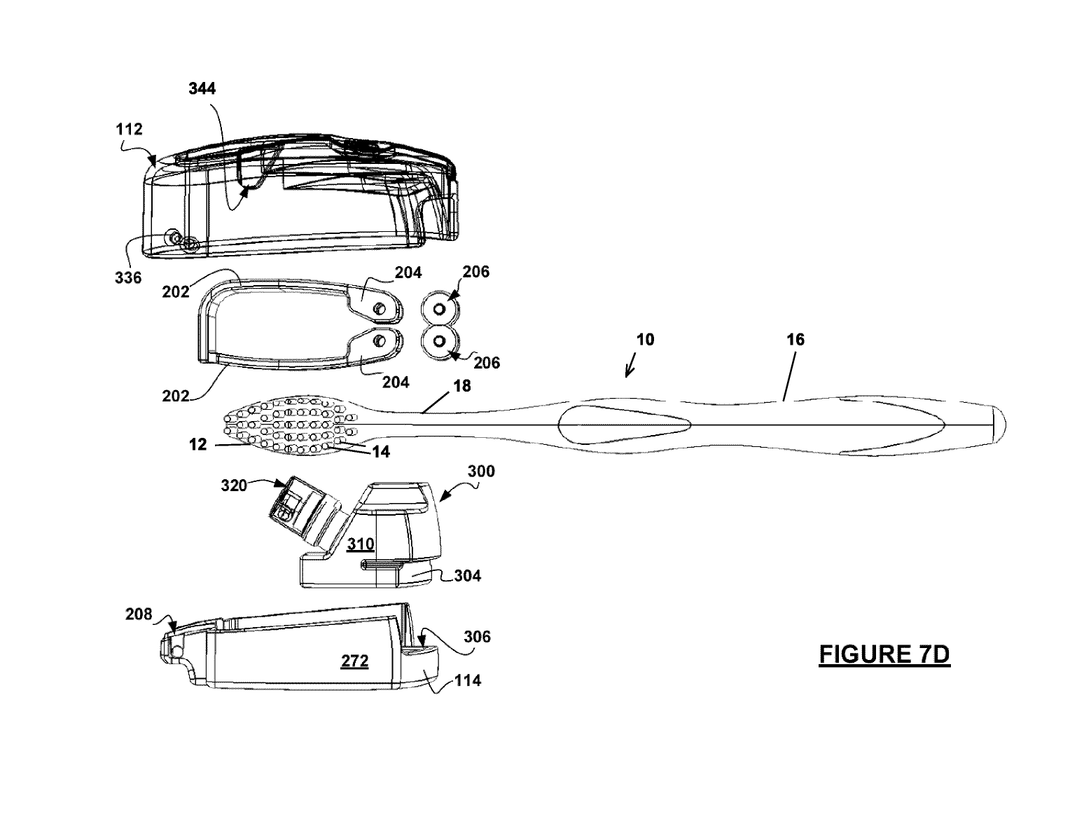

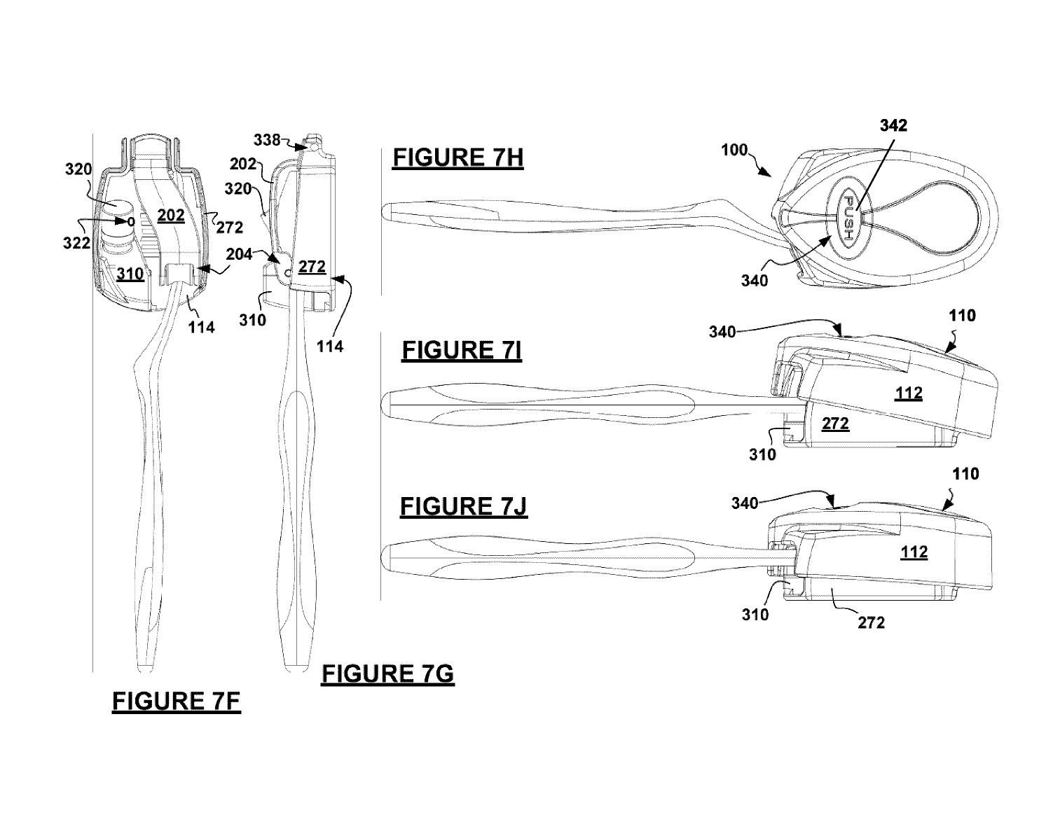

FIGS. 1A, 1B, 1C and 1D illustrate a toothbrush holder 100 in accordance with an embodiment of the proposed solution. Referring to FIG. 7D, there is also shown an example of an individual toothbrush 10 having a head 12, bristles 14 extending from the head 12, a handle 16 and a neck 18 between the head 12 and the handle 16.

In accordance with the embodiment of the proposed solution, the toothbrush holder 100 generally includes a toothbrush holder enclosure 110, or housing, a toothbrush retaining mechanism such as clamping member 200 and a reservoir for a sanitary agent 300. The housing 110 is configured to protect the toothbrush 10 inserted in the toothbrush holder; preferably the housing 110 is a splash-proof enclosure. An opening 102 is provided at the bottom of the enclosure 110 for passing a toothbrush therethrough. In the illustrated embodiments, the head 12 is removably insertable within the enclosure 110 through the opening 102 along a longitudinal axis of the toothbrush 10.

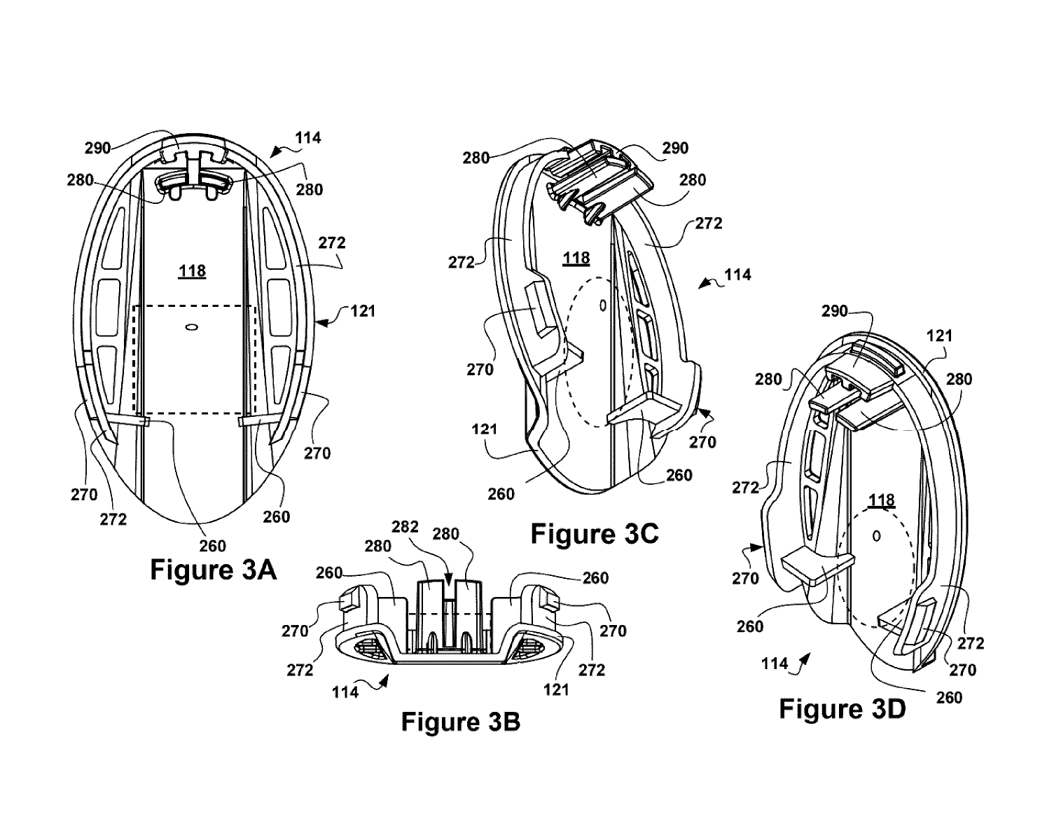

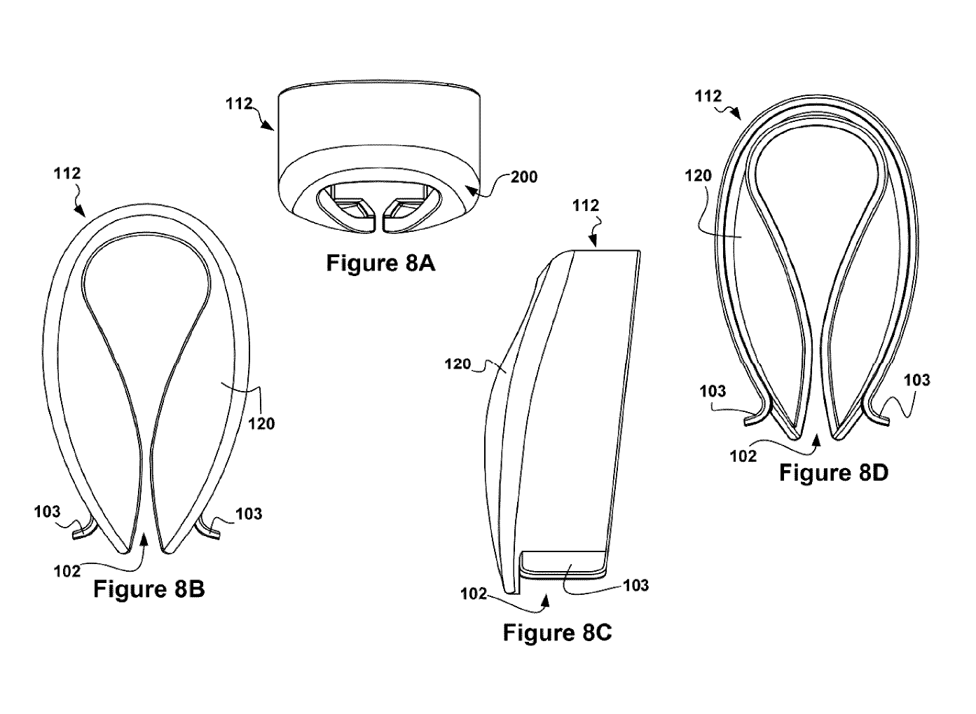

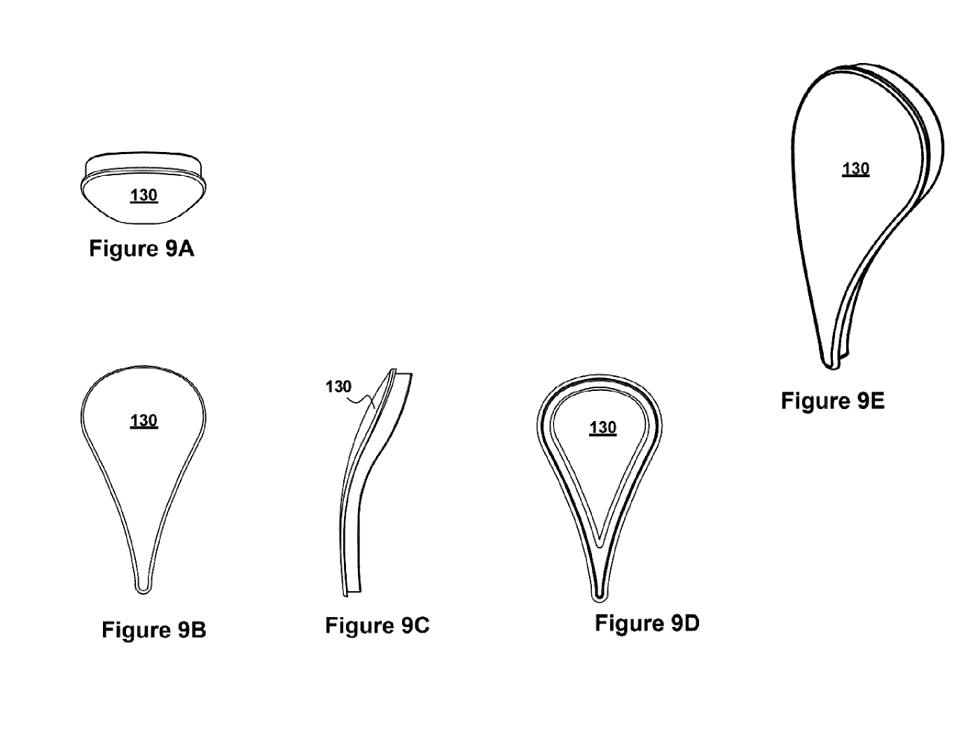

In accordance with a first implementation, the toothbrush holder enclosure 110 includes a front cover 112 and a back cover (not necessarily a plate) 114. FIG. 1B illustrates the front cover 112 of the enclosure 110, while FIGS. 1A, 1C and 1D illustrate both the front cover 112 and back cover 114, in a configuration in which the front cover 112 is mated to the back cover 114. For example, with reference to FIGS. 3A, 3B, 3C and 3D the front cover 112 and the back cover 114 can be configured to snap-fit and together or to slidingly engage each other. However the front cover 112 and the back cover 114 can be glued together for disposable configurations. Preferably, the front cover 112 is removable, for example in order to be cleaned in a dishwasher or by any other appropriate means of sanitation compatible with the material from which the front cover 112 is manufactured. Without limiting the invention, for a wall mounted configuration illustrated in FIGS. 1A and 1D the front cover 112 is further configured to expose the back plate 114 as little as possible, for example to prevent splashing. The bottom of the front cover 112 can form part in defining the enclosure opening 102. Without limiting the invention, FIG. 1C illustrates the front cover 112 and back plate 114 mating at an angle 116 to the vertical; in this first embodiment the back plate 114 having a general wedge shape. Further description of other details of the front cover 112 of this first embodiment are presented hereinbelow with reference to FIGS. 8A, 8B, 8C, 8D, 9A, 9B, 9C, 9D, and 9E. For clarity, the invention is not limited to a symmetric toothbrush holder enclosure 110; symmetric toothbrush holder enclosure 110 implementations are only illustrated for ease of description herein. An asymmetric toothbrush holder enclosures 110 are illustrated in FIGS. 7A and 7B.

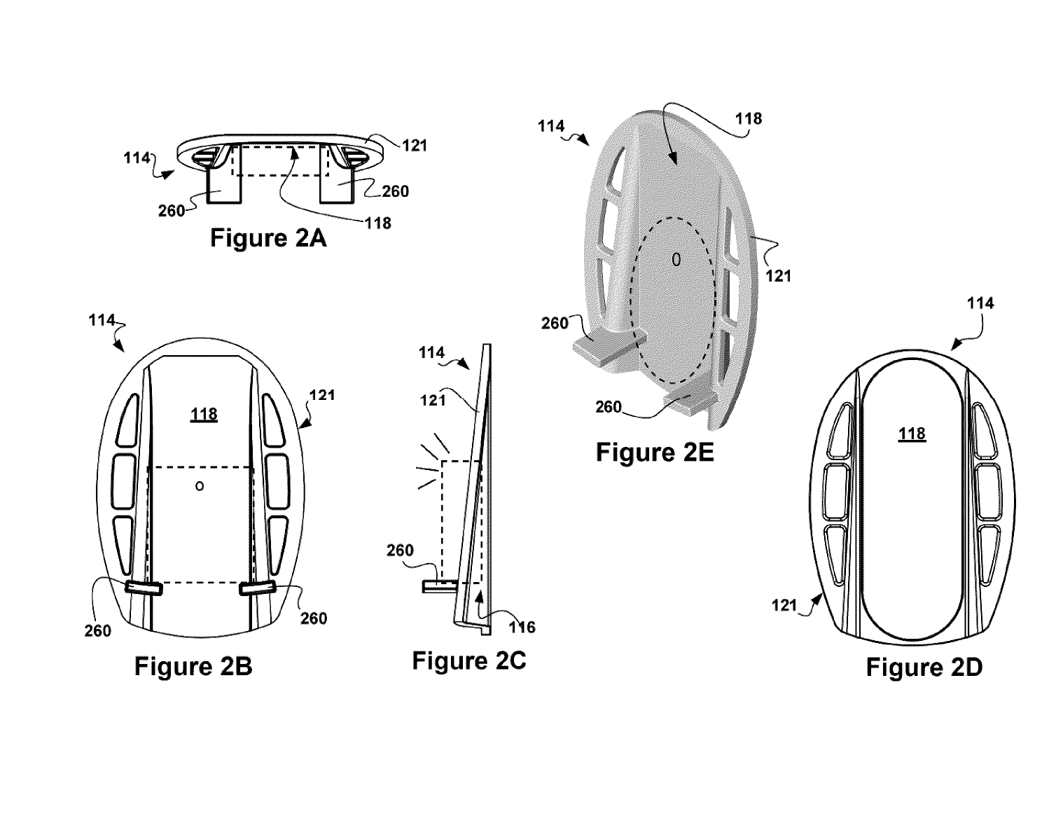

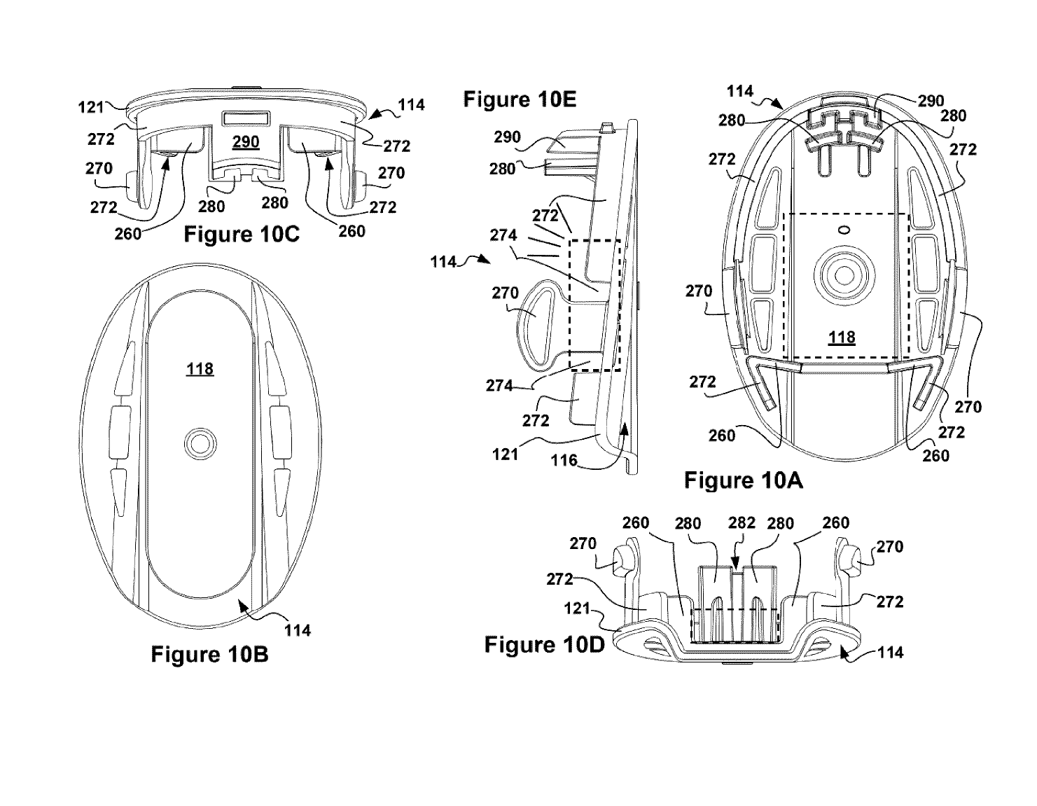

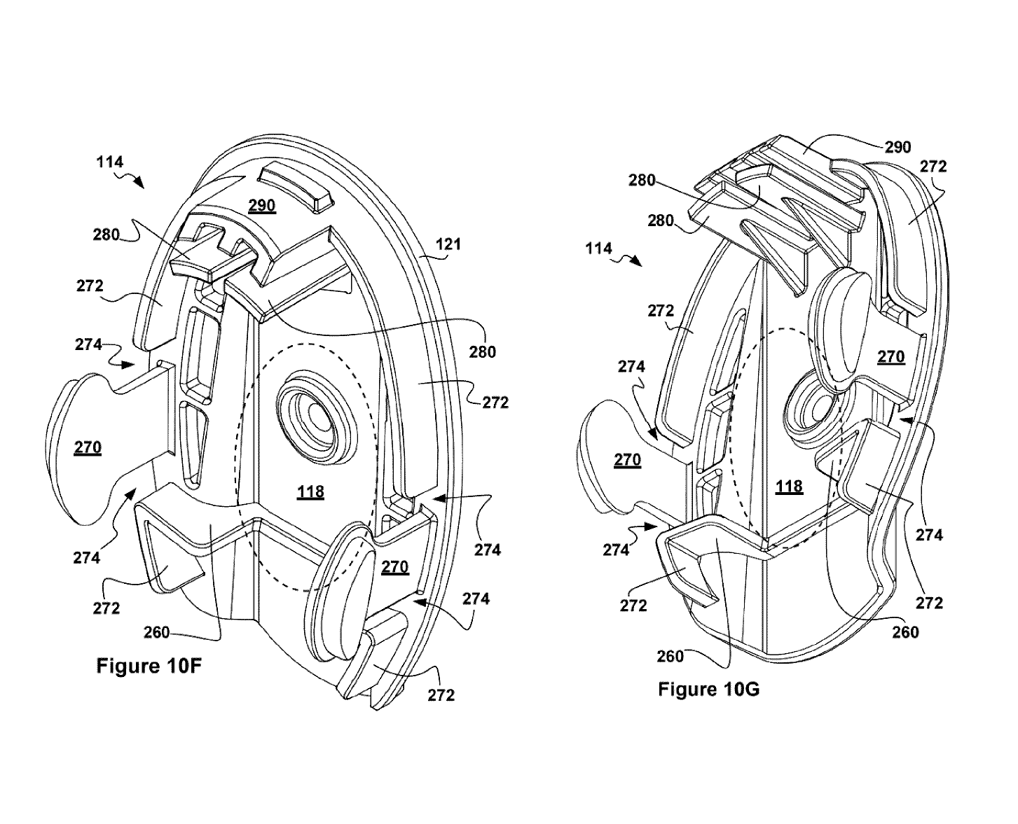

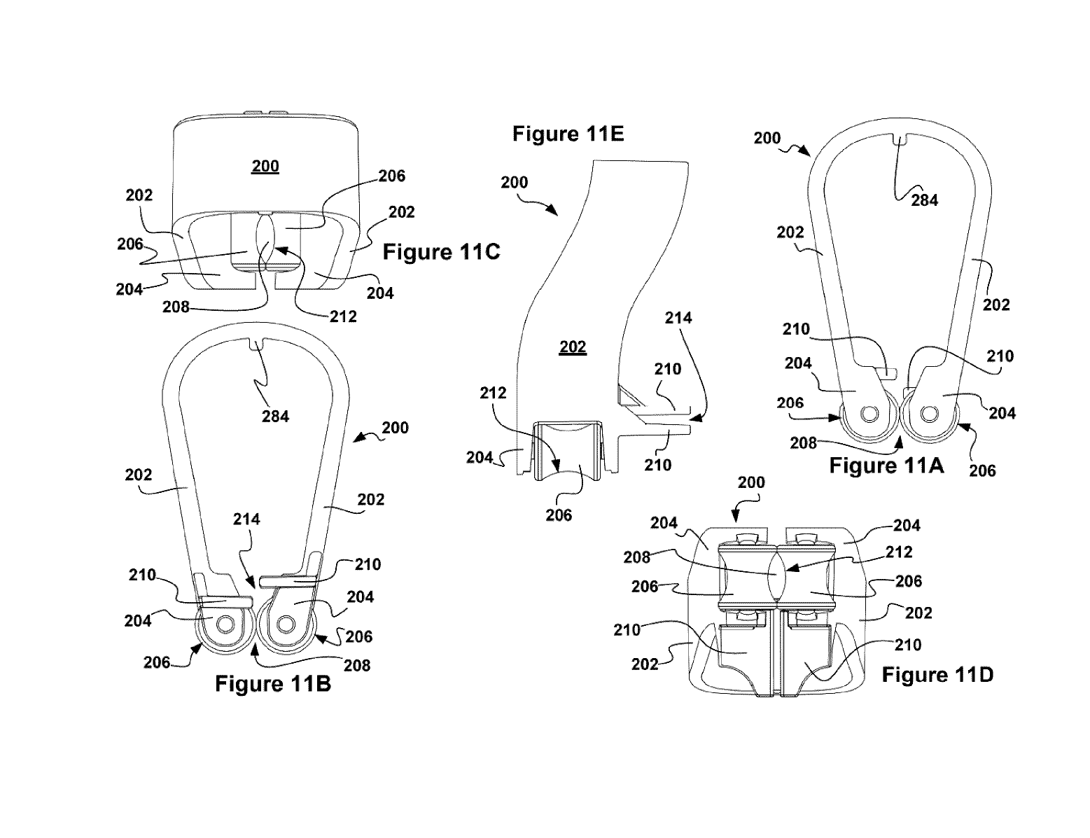

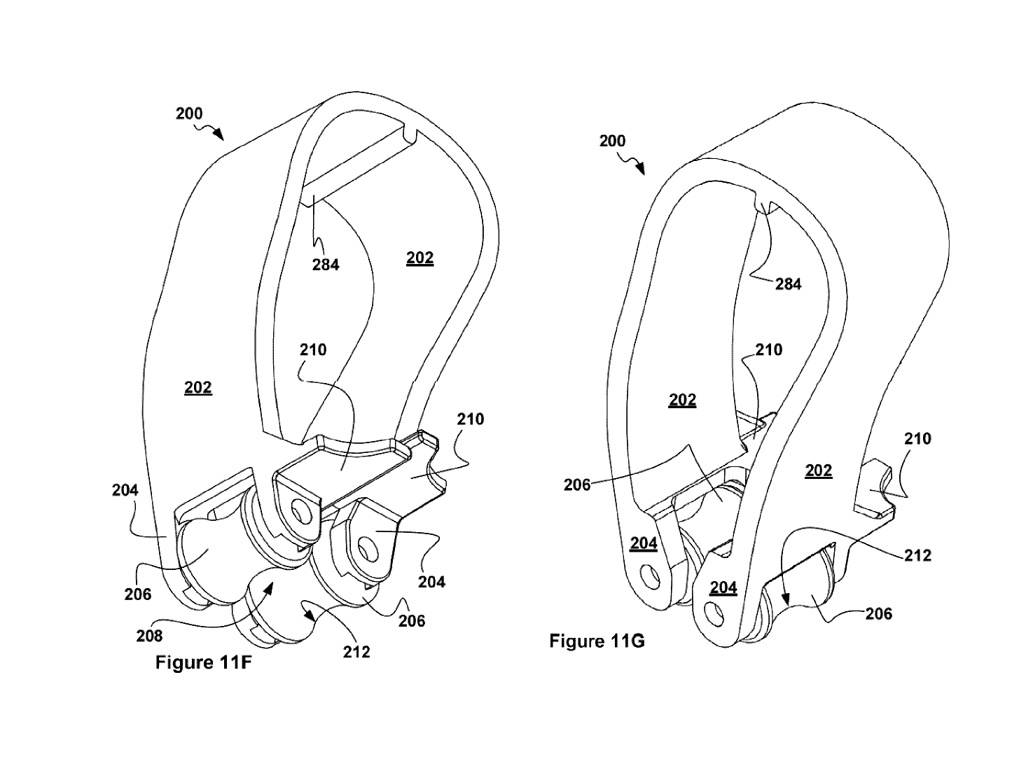

Returning to the back plate 114, FIGS. 2A, 2B, 2C, 2D, and 2E illustrate a back plate 114 in accordance with a first implementation the embodiment of the proposed solution. The back plate 114 has a generally flat shape, including a flat middle portion 118. The back plate 114 is configured to be affixed to a substantially vertical wall, preferably 15 semi-permanently for example on tile with an adhesive or permanently (with reference to FIGS. 10A, 10B, 10F and 10G on wood with fasteners not shown) such as screws and the like. A disposable toothbrush holder 100 can employ a suction cup fastener. Without limiting the invention, FIG. 2D illustrates an oblong central region in the flat middle portion 118 where an adhesive or two sided tape can be applied.

The back plate 114 of this implementation of the first embodiment has a peripheral edge 121 partially employed in providing the snap-fit with the front cover 112, wherein the front cover 112 has snap tabs configured to extend around and about the obround peripheral edge part of the back cover reducing back plate 114 exposure even further (not illustrated) than illustrated in FIGS. 1A, 1C and 1D. In accordance with another implementation, the snap tabs provide restricted sliding engagement between the front cover 112 and the back cover 114, for example between an non-actuated position FIG. 7I and an actuated position FIG. 7J. For some disposable implementations of the proposed solution, the front cover 112 can be glued to the back plate 114 around the obround peripheral rim 121 of the back plate 114. The bottom of the peripheral edge 121 of the back plate 114 can form part in defining the enclosure opening 102.

The invention is not limited to a discontinuous peripheral edge 121, FIGS. 3A, 3C and 3D illustrate a wholly ovaloid peripheral edge 121.

The back plate 114 is further configured to engage a toothbrush grip element (200) of the toothbrush holder 100. In accordance with an implementation of the first embodiment of the proposed solution, a pair of substantially horizontal support (bottom) tabs 260 extending forward with respect to the toothbrush holder enclosure 110 as illustrated and away from the back plate 114 is provided for at least one of the sanitizing agent dispenser (300) and the toothbrush grip element 200 to rest thereon. The invention is not limited to a pair of support tabs 260, to horizontal support tabs 260 or forward extending bottom support tabs 260. For certainty, the pair of symmetric support tabs 260 is illustrated only for ease of description, a single tab 260 can be sufficient as will be described hereinbelow with reference to the toothbrush grip element 200. One or more tabs 260 can alternatively extend sideways with respect to the orientation of the drawings (not shown). The back plate 114 can have a wedge shape (166) when viewed from the side as illustrated in FIG. 2C which orients the peripheral edge 121 of the back plate 114 at an angle 116 to the middle portion 118 of the back plate 114, for example for mounting the toothbrush holder 100 to a wall at an angle 116 to the wall to which the back plate 114 is affixed. As such, the support tabs 260 can be substantially perpendicular to the wall, or substantially perpendicular to a plane of the peripheral mating edge 121 of the back plate 114. For certainty the peripheral mating edge 121 of the back plate 114 need not lie in a plane, and the support tabs 260 can be angled towards the wall or angled to one side.

In accordance with a second implementation of the first embodiment of the proposed solution, FIGS. 3A, 3B, 3C and 3D illustrate a back plate 114 including snap tabs 270 for engaging a front cover 112 in snap-fit or in sliding engagement, and a different configuration for engaging a toothbrush grip element 200.

In accordance with the second implementation of the first embodiment, the snap-fit and/or restricted sliding engagement between the front cover 112 and the back plate 114 is provided by a short wall 272 extending forward from the back plate 114, not necessarily continuously, generally around the peripheral edge 121 of the back plate 114. The wall 272 in this second implementation enables improved snap-fit mating and/or sliding engagement, and can be manufactured out of a resilient material, for example a suitable polymer. The wall 272 can be discontinuous with notches 274 (For example illustrated in FIG. 10E, 10F and 10G) between non-bending portions 272 of the wall 272 and bending snap tabs 270 provided in the short wall 272. The snap-tabs 270 can include hook elements for engaging the front cover 112 to retain the front cover 112 in snap-fit engagement with the back cover 114 or to restrict sliding engagement between the front cover 112 and the back cover 114.

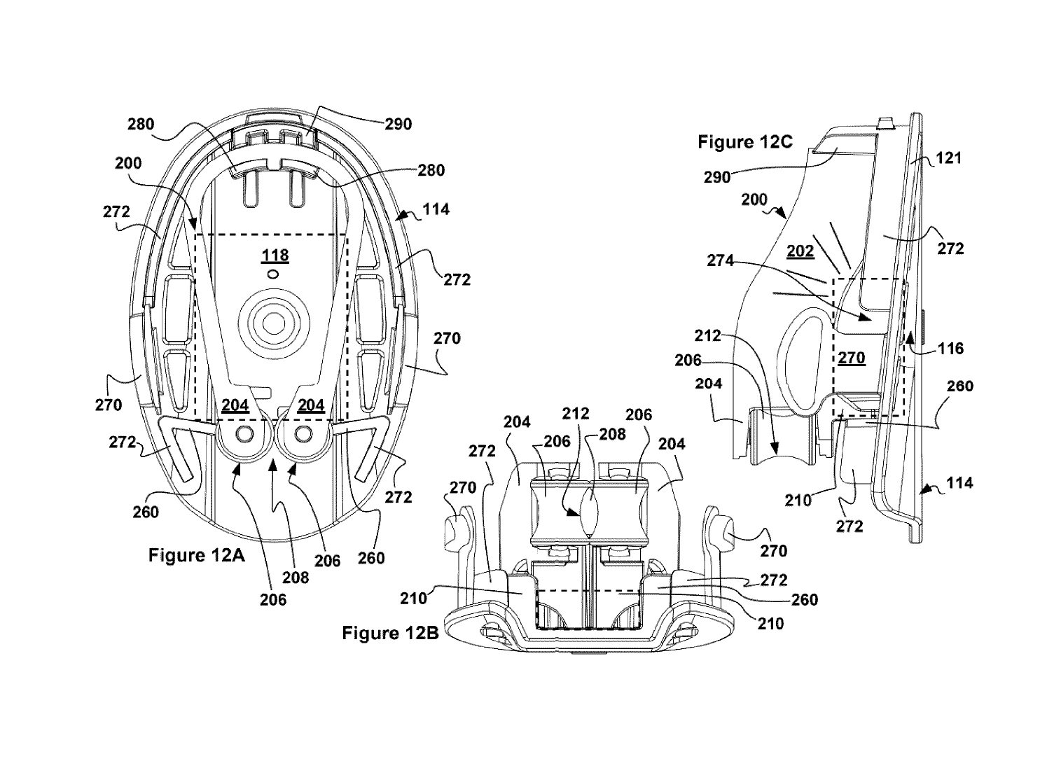

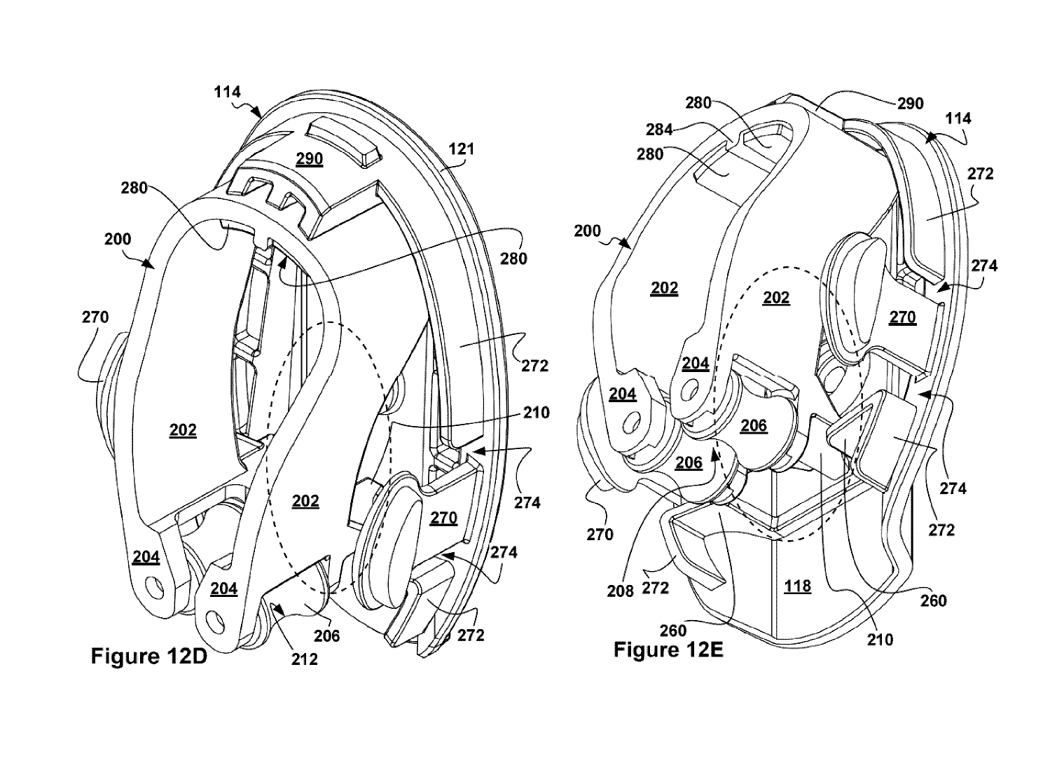

It is noted that the toothbrush is inserted through the enclosure opening 102 while the toothbrush is held in by the grip element 200—some form of alignment between the enclosure opening 102 and the grip element 200 can be beneficial though not necessarily required in every embodiment. In accordance with the second implementation of the first embodiment, support and alignment (260) is provided for the toothbrush grip element 200 and for the sanitizing agent dispenser 300. A pair of support tabs 260 is provided, however it is noted that the support tabs 260 illustrated in FIGS. 10A and 12A can be angled with respect to each other (towards, away, askew, etc.). Without limiting the invention, the orientation angle of at least one of the support tabs 260 can provide a way for the grip element 200 to return to a grip configuration, enhance the grip on the toothbrush, align the grip element 200 or any combination of thereof. Without limiting the invention, the orientation angle of at least one of the support tabs 260 can provide alignment for a sanitizing agent dispensing orifice 322 with respect to at least one of the grip element 200 and toothbrush holder enclosure 110.

Another support tab, for example the top support tab 280 illustrated, can be provided for example substantially at the apex of the back plate 114 and configured to provide further support for the grip element 200 and/or alignment. Without limiting the invention, alignment can be provided by the top tab 280 being slanted with respect to the back plate 114 and/or by a slit 282 therein (between a pair thereof) for an appropriately configured grip element 200 extending into the slit 282, for example a spine 284 on the grip element 200 extending into the slit 282. For certainty, symmetry of the support tab(s) 280 is only incidental and not a requirement of the invention.

While not required, left and right snap-tabs 270 extend out beyond the short wall 272 and are illustrated positioned about the bottom support tabs 260, for example enhancing the strength of the bottom support tabs 260 and/or providing further side alignment for the toothbrush grip element 200 and/or the sanitizing agent dispenser 300.

Another optional crown tab 290 is illustrated. The crown tab 290, either by itself or in combination with the toothbrush holder enclosure 110 provides an opposing force while the toothbrush is inserted into the toothbrush holder 100. The crown tab 290 can also counter the material deflection also employed in configuring the snap-tabs 270. When the crown tab 290 is not present the front cover 112 or the short wall 272 provides the opposing force while the toothbrush is inserted into the toothbrush holder 100. For certainty the crown tab 290 need not be crown shaped.

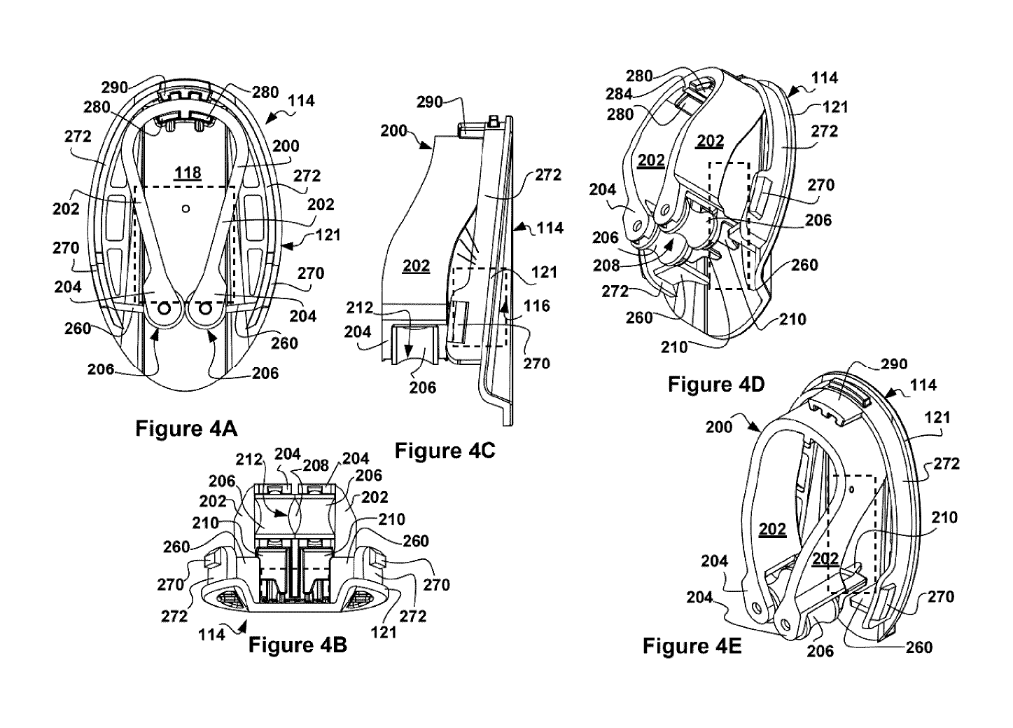

In accordance with the second implementation of the first embodiment of the proposed 20 solution, FIGS. 4A, 4B, 4C, 4D, and 4E illustrate a combination of: back plate 114, toothbrush grip element 200, and sanitizing agent dispenser 300. The grip element 200 cooperates with the back plate 114, for example, via at least one bottom support tab(s) 260, top support tab(s) 280 and optionally the crown tab 290. A grip element tab(s) 210 can rest on bottom support tab(s) 260 of the back plate 114. The illustrated grip element 200 includes a guide 284 cooperating with the slit 282 between top support tabs 280, without limiting the invention thereto. References to a “cover body” herein are intended to include the toothbrush holder enclosure 110 as a whole.

The toothbrush grip element 200 is configured to mechanically engage the toothbrush by clamping thereof, preferably the toothbrush head. In accordance with one way of using the combination, the top support tab(s) 280 (and crown tab 290 when present) securely engages the grip element 200, for example via snap-hooks or another separable engagement, and the toothbrush is inserted in the grip element 200 against the crown tab 290 and/or the wall 272 (which ever present) and extracted from the grip element 200 against at least one of the support tabs 280/260. In accordance with another way of using the combination, the top support tabs 280 operate as a sideways hook (not shown) on which the grip element 200 is slid sideways with respect to the orientation of the drawings.

For certainty the toothbrush grip element 200 engages the toothbrush, preferably the toothbrush head, and is can be removable from the enclosure for example for (general) sanitation purposes. For certainty, the invention is not limited to employing a removable toothbrush grip element 200, in a disposable toothbrush holder 100 the toothbrush grip element 200 may not be removable. Disposable toothbrush holders 100 may be used in the hotel industry, force majeure, emergency response, response to an epidemic or in a hospital or senior care/home setting.

FIG. 4C best illustrates the angled portion (116) of the back plate 114, in combination with the grip element 200, the angled portion (116) of the back plate 114 can account for toothbrushes having handles curved towards to wall when inserted to prevent the toothbrush handle from urging (the toothbrush grip element 200 and) the toothbrush holder 100 as a whole away from the wall. The dashed line rectangle/oval represents the general space occupied by the sanitizing agent dispenser 300.

The combination of the front cover 112, back plate 114 and grip element 200 is configured to substantially align the travel path of the toothbrush with the enclosure opening 102, and align the toothbrush bristles with a sanitation agent dispensing orifice 322 supplied with sanitation agent from the reservoir 310. FIG. 4B best illustrates ends 204 of the grip element 200 aligned with the enclosure opening.

FIGS. 7A to 7J illustrate a sanitizing toothbrush holder 100 having a sideways configuration as described herein below.

### Clip

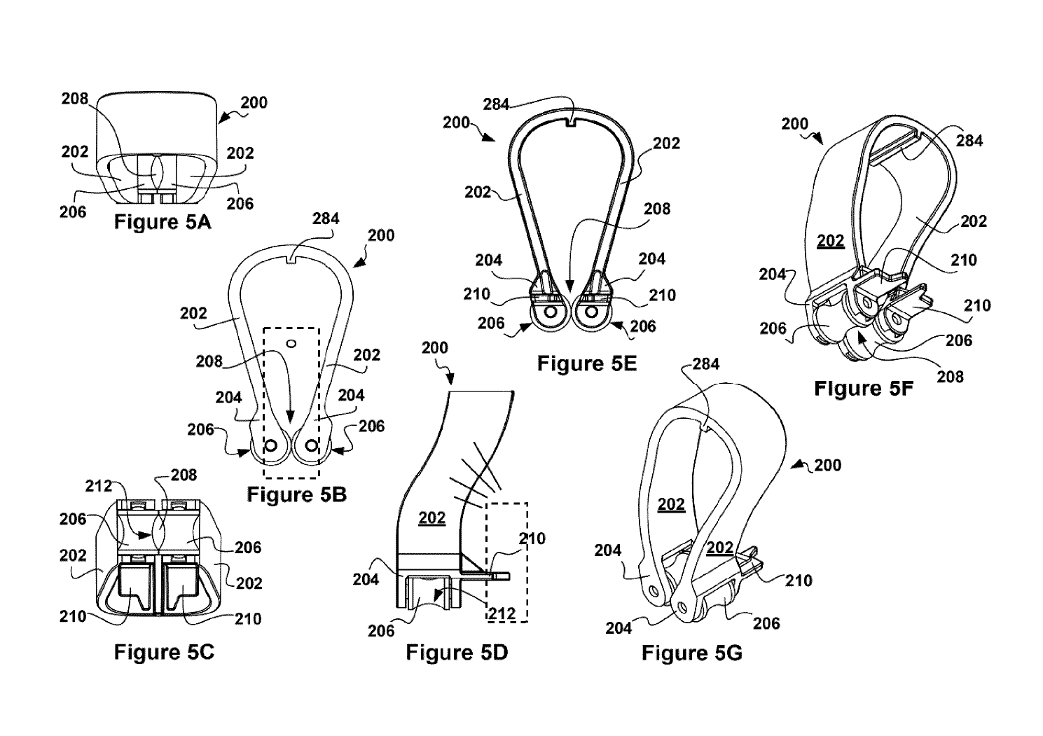

FIGS. 5A, 5B, 5C, 5D, 5E, 5F and 5G illustrate a first implementation of the toothbrush grip element 200 in accordance with the first embodiment of the proposed solution.

The toothbrush grip element 200 includes a clip (200) and optionally alignment element(s) (210) 284 for example as described above. For certainty, the spine 284 is present only in some implementations. As described hereinabove, the presence of the spine 284 does not imply a requirement. For the reminder of the description references to a “clip” include the toothbrush grip element 200.

The clip 200 includes two opposing grip members 202, biased towards each other, cooperating to engage (and retain) at least the toothbrush head. The clip 200 is configured to grip at least one of the toothbrush head and a toothbrush neck to hold the toothbrush in the toothbrush holder 100. The grip members 202 can be biased against each other being manufactured from a resilient material, for example an elastic material not limited to polymers. A metal clip 200 could also provide the necessary resilient grip. Form a manufacturing perspective, polymers can be injection molded into the form of the clip 200. The invention is not limited to a manufacturing technique.

The grip members 202 are separated by the insertion into, and conversely by the extraction out, of the head portion of the toothbrush.

In some embodiments the toothbrush is held in the toothbrush holder against the normal force of gravity. In other embodiments the toothbrush head is held in the toothbrush holder 100 spaced apart (away) from the toothbrush enclosure 110. Preferably the engagement area between the toothbrush as a whole and the toothbrush holder 100 as a whole is minimized. Preferably engagement is reduced to grip members' 202 ends 204.

With reference to the first clip implementation, chiefly illustrated in FIGS. 4A, 4B, 4C, 4D, 4E, 5A, 5B, 5C, 5D, 5E, 5F and 5G, and without limiting the invention thereto, grip member ends 204 employ rollers 206. Each roller 206 is configured to journal around a corresponding pivot providing rolling mechanical engagement between the clip 200 and the toothbrush. The head portion of the toothbrush can slide over (at between) the rollers 206 until the toothbrush snaps into grip due to the return pinching force of the clip 200. In some implementations the toothbrush snaps into grip when the rollers 206 engage the neck portion of the toothbrush.

For certainty, the invention is not limited to both grip members 202 being resilient. A single resilient grip member 202 impinging against a stiff grip member (202) would work. For example, an implementations illustrated in FIG. 7B employed a resilient grip member 202 impinging against the back cove 114 which is stiff.

Preferably the rollers 206 form a channel-like guide 208 therebetween for the toothbrush head and/or neck. For example the rollers 206 can include a circumferential notch 212 defining the channel-like guide 208 therebetween. When only one roller 206 is employed against a stiff opposing member 202, the single circumferential notch 212 of the single roller can provide the channel-like guide 208. The circumferential notches 212 (either singly or in combination) present a concave depression at the bottom of the toothbrush holder 100 to the toothbrush head leading into the channel-like guide 208 between the gripping members 202 such that each roller 206 engages the toothbrush head using saddle-like region.

For certainty, the invention is not limited to the use of two rollers 206. More than two rollers 206 can be employed (not shown) which need not engage the neck of the toothbrush. A single roller 206 impinging against the other grip member 202 would also work. For example, employing rollers 206 enables use of toothbrushes with rubberized head sides.

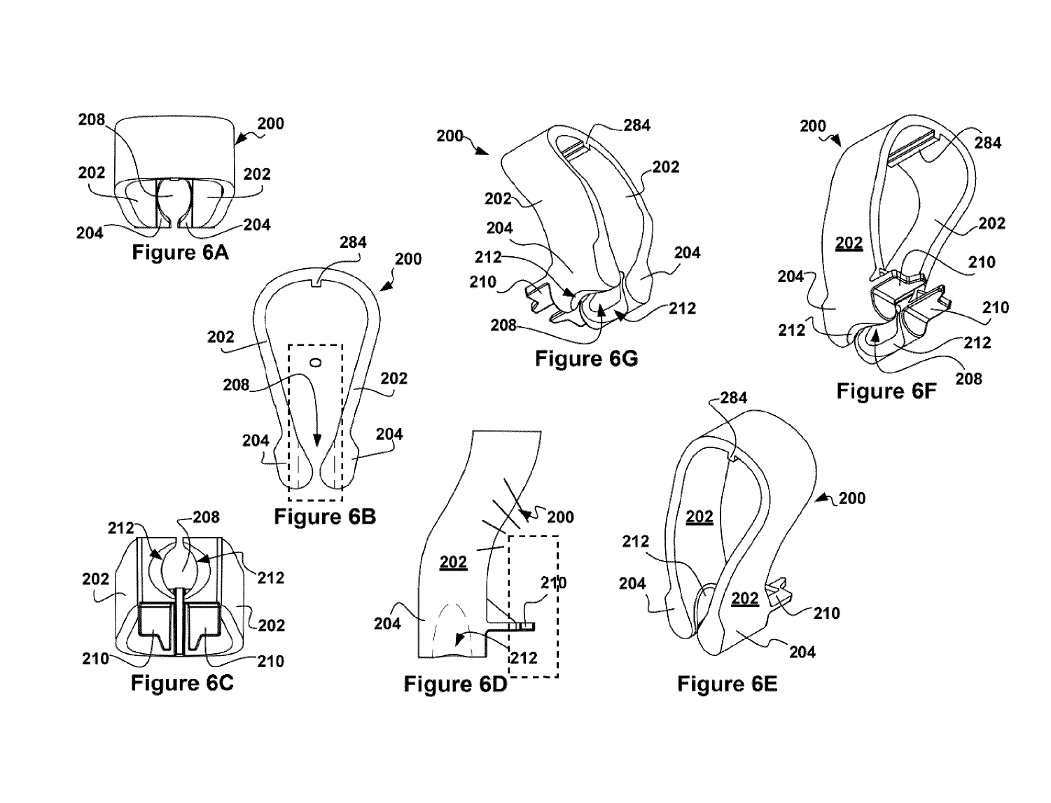

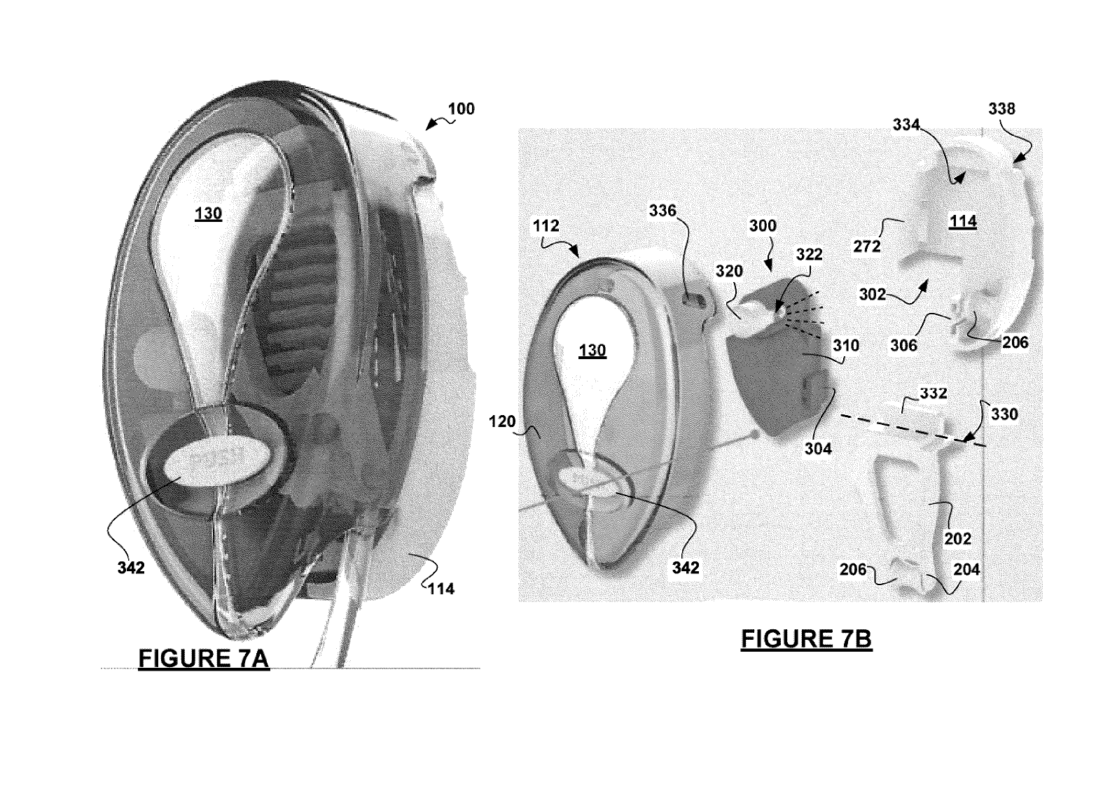

FIGS. 6A, 6B, 6C, 6D, 6E, 6F and 6G illustrate a second implementation of the toothbrush grip element 200 in accordance with the first embodiment of the proposed solution. For brevity description of structures and elements similar to the first toothbrush grip element 200 implementation is not repeated. The second clip 200 implementation does not employ rollers (206) per se, instead grip member ends 204 include opposing notches 212 defining the channel-like guide 208 between them. The notches 212 are configured to engage at least the toothbrush head via sliding mechanical engagement. The notches 212 are further flared away from each other presenting to an incoming toothbrush head a concave depression leading into the channel-like guide 208 such that each notch 212 engages the toothbrush head using saddle-like region. Similar variations can be employed as with rollers 206, variations in the number of notches 212 and material resilience of the grip members 202.

Preferably the clip 200 includes at least one tab 210 extending from said ends 204 15 generally away from the clip ends 204 in a desired orientation of the bristles of the toothbrush. For example, FIGS. 4A, 4B, 4C, 4D, 4E, 5A, 5B, 5C, 5D, 5E, 5F, 5G (and 6A, 6B, 6C, 6D, 6E, 6F, 6G) illustrate a pair of clip tabs 210 extending towards the back of the toothbrush holder 100. However, in accordance with the proposed solution, the pair of clip tabs 210 extend towards the dispensing orifice 332 of the reservoir element 310 for inserting a toothbrush in with the bristles towards the orifice 322.

The clip tabs 210 form a slit 214 between them. In accordance with the proposed solution, the clip tabs 210 are configured to prevent the toothbrush head from being inserted into the clip 200 while the toothbrush head can be inserted between the clip rollers 206 forcing a particular orientation of the toothbrush. The slit 214 allows the bristles to pass between the tabs 210.

While FIGS. 4A, 4B, 4C, 4D, 4E, 5A, 5B, 5C, 5D, 5E, 5F, 5G (and 6A, 6B, 6C, 6D, 6E, 6F, 6G) illustrate clip tabs 210 generally perpendicular to the direction of travel of the toothbrush head into the clip 200, the invention is not limited to blocking toothbrush travel. Alternatively, the clip tabs 210 can be configured to present a convex overall surface to the incoming toothbrush head deflecting the toothbrush head away. As well the clip tabs 210 can be in the same plane angled away or askew (FIGS. 11A, 11B, 11E and 11F) with respect to the direction of travel of the toothbrush head.

At least one clip tab 210 may be used to space the bristles a distance apart from the dispensing orifice 322 of the reservoir 300. Without limiting the invention, the clip 200 or a clip tab 210 can act to cause sanitizing agent to be dispensed or to act as actuator either directly or indirectly to trigger release of sanitizing agent via the orifice 322. For example, a clip tab 210 can be configured to (mechanically) impinge on an aspect of the reservoir element 300 to cause the release of sanitizing agent.

FIGS. 4A, 4B, 4C, 4D, 4E, 5A, 5B, 5C, 5D, 5E, 5F, 5G, 6A, 6B, 6C, 6D, 6E, 6F and 6G illustrate a clip 200 body deflected (top) and extending towards the back plate 114 in the direction of the bristles to space the bristles away from the orifice 322 located 15 between the clip 200 and the back plate 114. However the invention is not limited to spacing the bristles away from the back plate 114 via tab 210 tips. For certainty the invention is not limited to the use of clip tabs 210 to orient the toothbrush in the toothbrush holder 100.

FIGS. 7A and 7B illustrate another implementation of the sanitary toothbrush holder 100 in accordance with another embodiment of the proposed solution. FIG. 7A illustrates the assembled toothbrush holder 100 with a toothbrush lodged therein with the toothbrush bristles directed sideways. FIG. 7B illustrates an exploded view of the toothbrush holder 100 illustrated in FIG. 7A. For brevity description of structures and elements similar to the above embodiments is not repeated.

With reference to FIG. 7B, notably one of the rollers 206 is provided on the back cover 114 wherein the wall 272 serves to stiffen the back cover 114 to reduce compliance of the back cover 114. The opposing roller 206 is provided on a resilient member 202 configured to pivot about an axis 330. A tab 332 is configured to fit under a groove 334 in the top of the back cover 114 to provide alignment between the resilient member 202 and the back cover 114.

Notably the back cover 114 includes is not wholly ovaloid (in the sense illustrated in FIG. 7B) having a notch 302 in the lower left. The notch 302 is configured to provide a guide for a replaceable sanitizing cartridge 300 providing a reservoir 310 holding sanitizing agent. A dispense trigger 320 is provided in the cartridge 300. For example, in accordance with one implementation FIG. 7B illustrates the dispense trigger 320 spray button having a dispense orifice 322. The sanitizing agent in the reservoir 310 can be under pressure without limiting the invention thereto. Solely as the cartridge 300 and toothbrush are retained in the toothbrush holder 100 in side-by-side relationship, the dispense orifice 322 is directed sideways with respect to the toothbrush holder enclosure 110 towards the toothbrush bristles (see FIG. 7A). Without limiting the invention thereto, an additional tab 304 can be provided in the cartridge 300 configured for slidable mechanical engagement with a guiding and orienting channel 306 on the back cover 114. For example, for wall mountable implementations, the front cover 112 includes a hinge pin 336 configured to pivot in a groove 338 on the back of the back cover 114. While not required for the wall mountable implementation, the front cover 112 may include a depression 340 indicating a dispense actuation region which can be depressed by the user of the device. For travel implementations, the depression 340 can contain therein a button 342 for causing sanitation agent to be dispensed, the cover 112 acting to protect the actual button 342 from accidentally triggering release of sanitation agent. As explained further below, the underside of the button 342 can have at least one actuating finger 344 that is configured to push a trigger button 320 of a pump 312.

In accordance with the proposed solution, the sanitation toothbrush holder 100 includes release means for causing controlled dispensation of sanitation agent. In some implementations, the release means includes a release trigger, for example including a button causing the reservoir to dispense a measured amount of sanitation agent.

In accordance with a wall mountable implementation illustrated in FIGS. 7A and 7B, the front cover 112 (or actual button 342), when depressed, impinges on the dispense trigger 320 for dispensing sanitation agent from the reservoir 310 through the dispense orifice 322. For example, with the toothbrush secured by the resilient member 202 between rollers 206, if the sanitation agent is a fluid, the spray button 320 can cause sanitation agent to spray out from the dispensing orifice 322 configured as a nozzle onto the toothbrush bristles. The spray button 320 can be a push button of a pump spray dispenser. As another example, prior to inserting the toothbrush into the sanitizing toothbrush holder 100, the dispense trigger 320 can cause sanitation agent in the form of gel or paste to issue from the dispensing orifice 322 forming a globule about the dispensing orifice 322, the globule extending a distance towards the rollers 206. Inserting the toothbrush head into the toothbrush holder 100 past the rollers 206 causes the bristles to break the meniscus of the sanitizing agent globule. The bristles wick up the sanitation agent for example by capillary action.

FIG. 7C illustrates a perspective view of an open sanitizing toothbrush holder for loading in a sanitation agent dispenser cartridge 300 in accordance with another implementation of the proposed solution. FIG. 7D illustrates an exploded view of a sanitizing toothbrush holder having a sanitation agent dispenser cartridge (in relation to a toothbrush). The clip 200 is removed and the dispenser cartridge 300 is shown prior to being slid into mechanical engagement of the tab 304 and channel 306. In accordance with the illustrated implementation, however without limiting the invention thereto, the front cover 112 is rigid, hinged via hinge pin 336 into the hinge depression 338 of the back cover 114, and the front cover includes actuator finger(s) 344 configured to impinge on the spray button 320 to cause controlled dispensation of sanitation agent from the reservoir 310.

FIG. 7E illustrates a perspective view of the sanitizing toothbrush holder without the front cover as illustrated in FIG. 7C however with the clip 200 and a toothbrush retained therein. Notably, the spray nozzle 322 is directed sideways with respect to the orientation of the toothbrush holder 100 towards the bristles of the toothbrush as retained in the clip 200. FIGS. 7F and 7G illustrate respectively a top and right side view of the configuration of the sanitizing toothbrush holder without the front cover illustrated in FIG. 7E. FIG. 7H illustrates a top view of the assembled sanitizing toothbrush holder illustrated in FIGS. 7C, 7D, 7E, 7F and 7G with the front cover 112 attached.

FIGS. 7I and 7J illustrate right side views of the sanitizing toothbrush holder in operation between a normal (non actuated, rest) position in FIG. 7I and an actuated position in FIG. 7J. In the actuated position, the actuator finger(s) 344 illustrated in FIG. 7C impinges on the spray button 320 causing a controlled amount of sanitation agent to be dispensed via the nozzle 322 towards the toothbrush head.

In other implementations (not shown), the release means includes deforming the enclosure to cause the reservoir to dispense a measured amount of sanitation agent. In further implementations (not shown), the release means includes deforming the reservoir to dispense a measured amount of sanitation agent. In further implementations (not shown), the release means includes deforming the clamp to cause the reservoir to dispense a measured amount of sanitation agent. In yet other implementations, the release means includes ratcheting means configured to cause the reservoir to deform to dispense a measured amount of sanitation agent. In some implementations the ratcheting means include a ratchet actuated by the insertion of the 20 toothbrush head into the toothbrush holder. In other implementations the ratcheting means include at least a roller having a ratcheting gear actuated by the insertion of the toothbrush head into the toothbrush holder.

### Front Cover

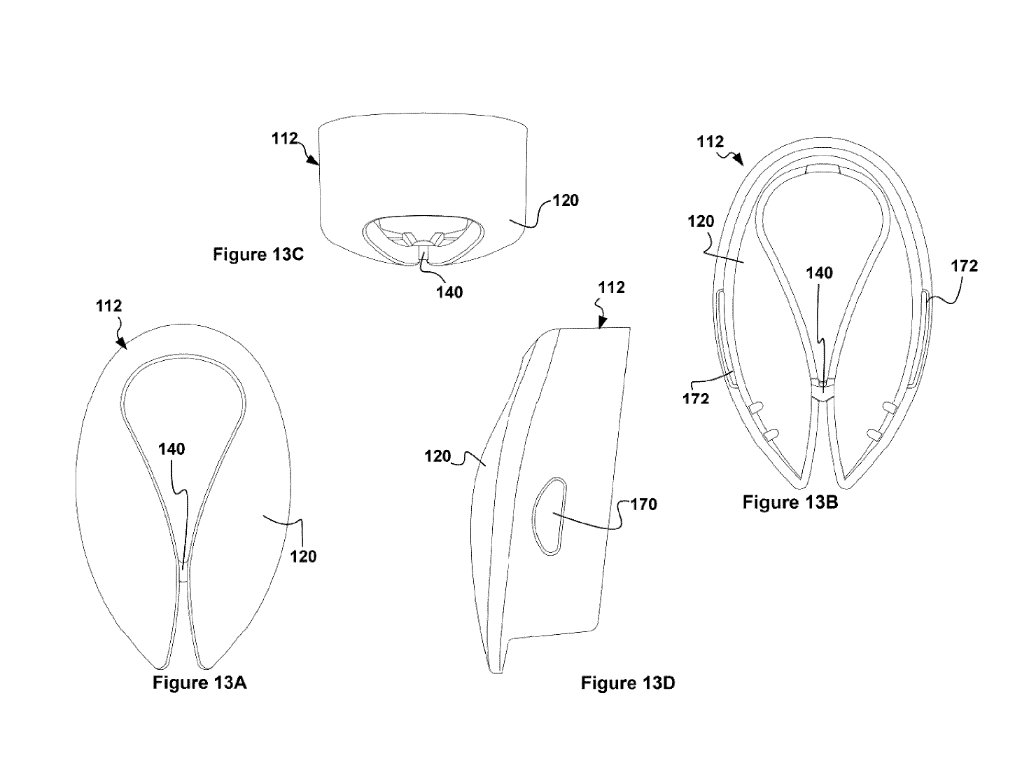

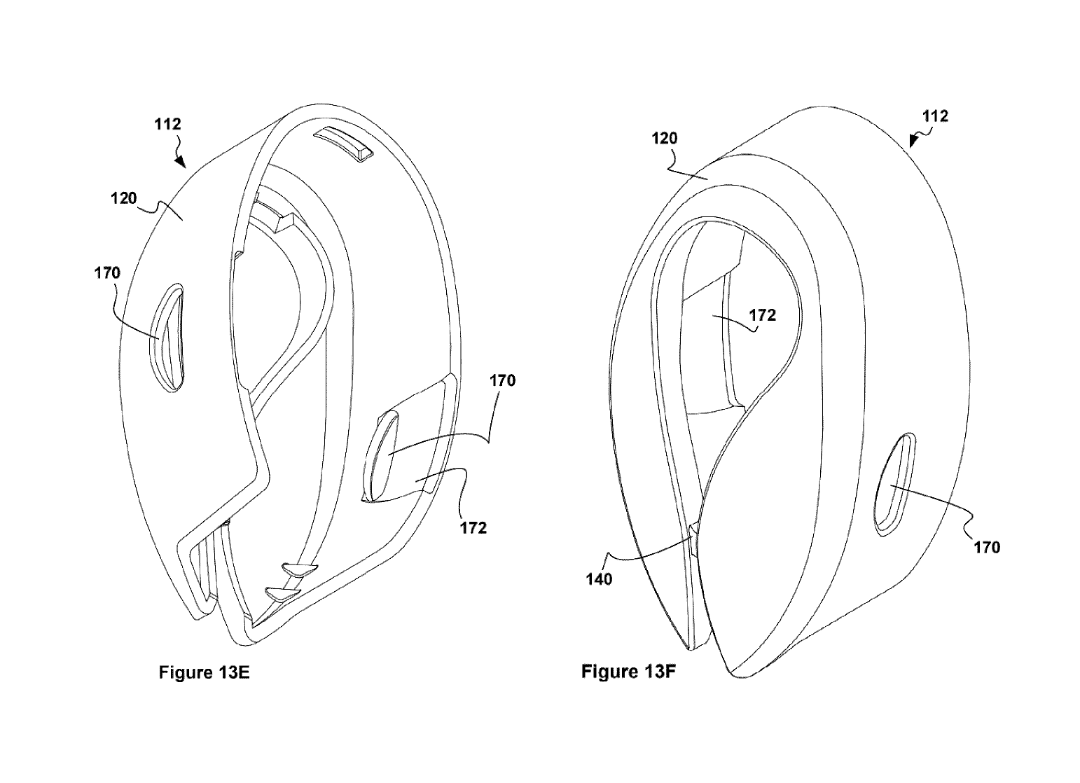

Returning to the description of the front cover 112, the front cover is not necessarily a single piece. FIGS. 8A, 8B, 8C and 8D illustrate a first front cover element 120 generally but not necessarily opaque configured to engage the back plate 114 as described hereinabove. In the implementation illustrated in FIG. 7A the front cover 112 is see through, and can be transparent. Preferably the cover 112, as illustrated in FIGS. 13B, 13D, 13E and 13F, engage the back plate 114 via a snap-fit engagement 270 as described herein above.

Preferably the front cover 112 includes flared sides 103 best illustrated in FIGS. 1B, 5 1C, 1D, 8B, 8C and 8D providing further guidance for the insertion of the toothbrush head into the toothbrush holder 100, but the flared sides 103 are not required.

The front cover 112 can be provided with a window 130 into the toothbrush holder enclosure 110. FIGS. 9A, 9B, 9C, 9D and 9E illustrate a second front cover element 130, a window. For example the front cover window 130 can be tear drop shaped and can have features of shape and configuration adapted for snap-fit into the first front cover element 120.

### Variant

In accordance with a fourth implementation of the first embodiment on the proposed solution FIGS. 10A, 10B, 100, 10D, 10E, 10F and 10G illustrate a reusable sanitary back plate 114. Features in common with the back plate 114 illustrated in FIGS. 3A, 3B, 3C and 3D are not repeated.

The back plate 114 has an overall ovaloid shape with an interrupted peripheral wall 272. Via material choice, for example a polymer, and/or material thickness freely extending snap tabs 270 can be configured to provide more bending than the peripheral wall 272 in engaging the front cover 112. The peripheral wall 272 can be configured to provide improved rigidity in engaging the front cover 112, whereas the snap tabs 270 act to pull the front cover 112 shut towards the back plate 114.

As illustrated in FIG. 10B an obround region 118 for affixing a double sided tape or an adhesive is provided on the back surface of the back plate 114, as well a reinforced opening is provided, as best illustrated in FIGS. 10A, 10B, 10F and 10G, for receiving and engaging a fastener or a suction cup (not shown) for secure engagement with a bathroom wall.

In accordance with the fourth implementation, the support tabs 270 are reinforced. The bottom support tabs 260 are integral with the peripheral wall 272 as best observable in FIGS. 10A, 10F and 10G. As best illustrated in FIGS. 10A, 10D and 10G the top support tabs 280 are reinforced via spines. With the top cover 112 hinged (336/338 not shown) on the back plate 114, the support tabs 270 can be configured to retain the top cover 112 in the non-actuated (FIG. 7I) position and to provide sliding engagement of the top cover 112 along the tabs 270 towards the actuated position (FIG. 7J).

FIGS. 11A, 11B, 11C, 11D, 11E, 11F and 11G illustrate a reusable sanitary toothbrush grip element 200 or clamp 200. Features in common with the clamp 200 illustrated in FIGS. 5A, 5B, 5C, 5D, 5E, 5F and 5G are not repeated.

The clamp 200 employs a pair of toothbrush grip element tabs 210 reinforced by corresponding spines best illustrated in FIGS. 11B, 11E and 11F. It is noted that the clamp tabs 210 are not in the same plane as illustrated in FIGS. 11A, 11B, 11E and 11F.

FIGS. 12A, 12B, 12C, 12D, and 12E illustrate an assembled reusable sanitary toothbrush grip element 200 and back plate 114 combination. Features in common with the combination illustrated in FIGS. 4A, 4B, 4C, 4D, and 4E are not repeated.

FIGS. 13A, 13B, 13C, 13D, 13E and 13F illustrate a reusable sanitary front cover 112/120. Features in common with the front cover 120 illustrated in FIGS. 8A, 8B, 8C and 8D are not repeated.

The front cover 120 lacks flared lateral walls (FIGS. 13A, 13B and 13E) while a 30 reinforcing bridge 140 is provided between the two sides forming the window (130). The bridge 140 best illustrated in FIGS. 13A, 13B, 13C and 13F can also be configured to participate in securing the window 130 as will be described herein below. A number of reinforcing spines are visible in FIGS. 13B and 13E providing improved rigidity around the bottom opening of the housing 110. Snap fit openings 170 for mechanically engaging the snap fit tabs 270 of the back plate 114 are best illustrated in FIGS. 13B, 13D, 13E and 13F. The snap fit tabs 270 are guided by channels 172 best illustrated in FIGS. 13B, 13E and 13F. Channels 172 can provide controlled slidable engagement on the front cover 112 between the front cover 112 and the back cover 114.

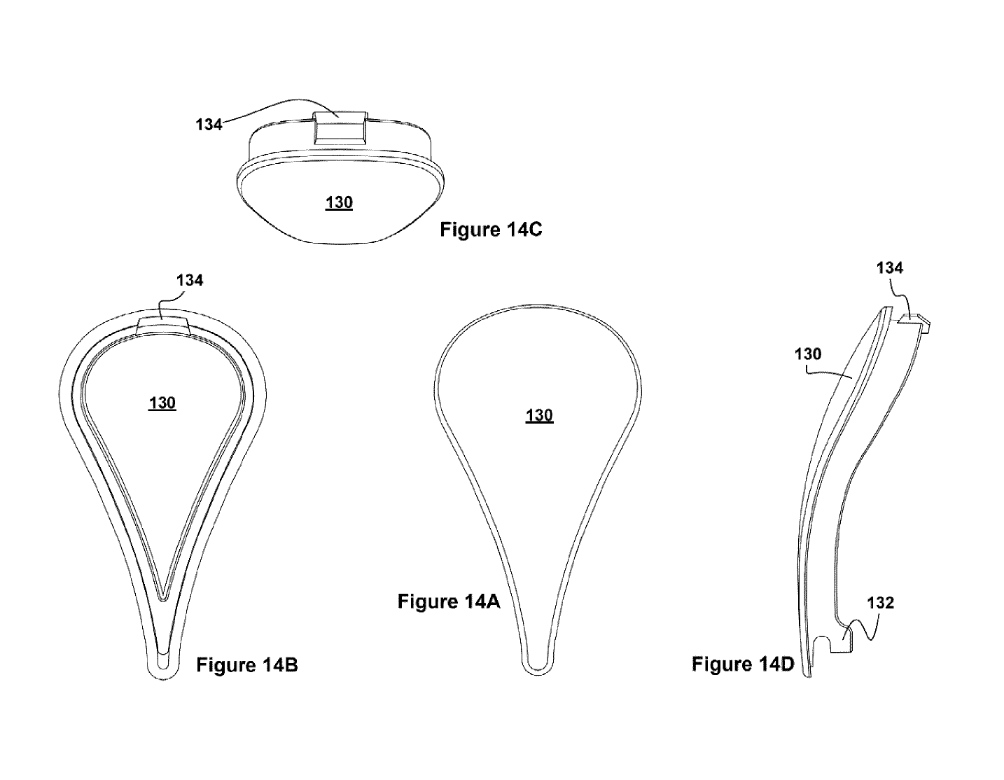

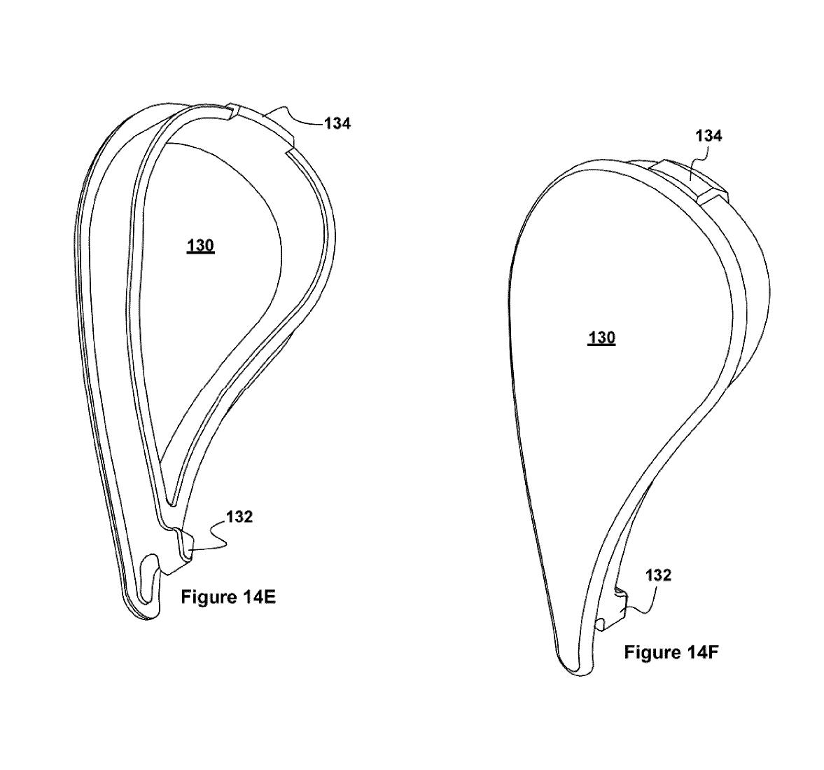

FIGS. 14A, 14B, 14C, 14D, 14E and 14F illustrate a reusable sanitary front cover window 130. Features in common with the front cover window 130 illustrated in FIGS. 9A, 9B, 9C, 9D and 9E are not repeated.

Notably the window 130 employs a bottom hook 132 for engaging the bridge 140 and a top snap fit bump 134 for engaging to top of the front cover 120. Both hook 132 and 15 bump 134 act to releasably retain the window 130 in snap fit engagement with the front cover 120.

While the toothbrush holder can be a clip with rollers for any kind of toothbrush head, in accordance with another variant if the toothbrush head has a predetermined shape then the toothbrush holder can be custom shaped to match the toothbrush head. The invention is not limited to a specific attachment for the toothbrush holder e.g. magnet, specially shaped slot, etc. would also work.

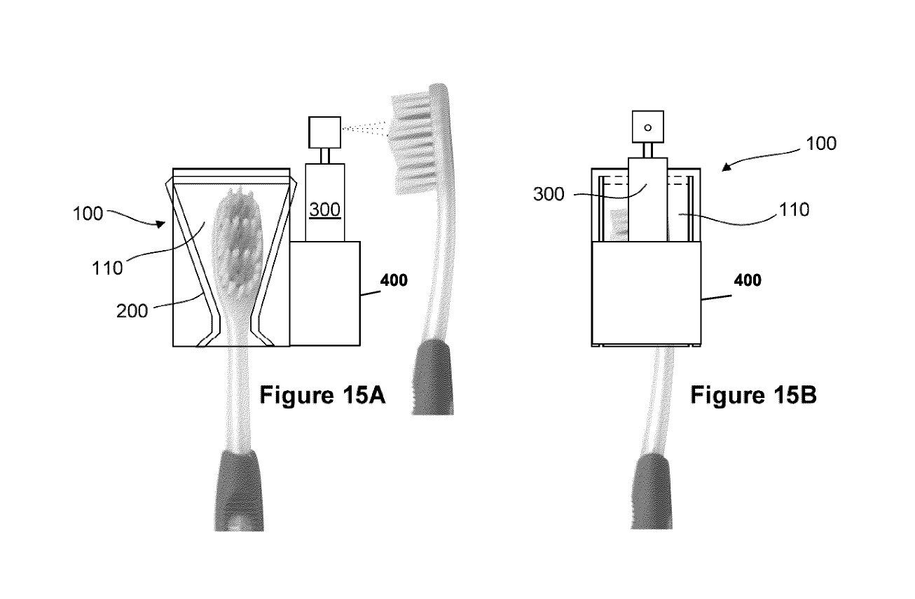

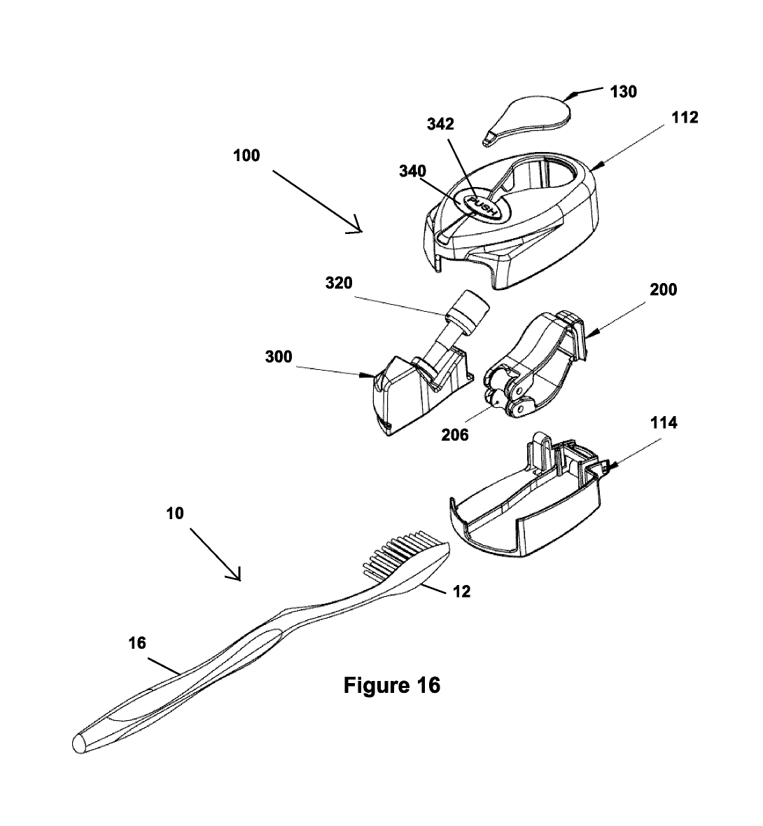

In the embodiment of FIGS. 15A and 15B, there is shown an example of a toothbrush holder 100 in which the sanitization agent dispenser 300 is a small spray unit that is held in a mounting 400 connected to an external side of the holder 100 so that the bristles of the toothbrush can be sprayed as illustrated in FIG. 15A with the agent. When the bristles are wet, the agent can be more easily wicked into the bristles. Thus, the user can rinse the toothbrush after brushing, and then apply the sanitization agent to the bristles using the spray device, and then insert the toothbrush head into the enclosure 110. In the embodiment shown, the sides of the enclosure are resilient clip members 200 that hold the head within the enclosure while guiding the head. The bristles do not, absent significant twisting force, contact the inside of the enclosure. While the clip members illustrated in FIG. 15A have no rollers or contour shaped to guide the head to prevent twisting, it will be appreciated that such elements can be provided, if desired.

The spray device can be replaced or refilled when empty. As described above, the small quantity of fluid contained in the applicator device can last for about a month of single person use. Such a holder 100 can be wall-mounted, or used for storage in a travel bag, on a counter or in a drawer, as desired.

While a spray unit is illustrated, it is also possible for a small pump in the dispenser 300 to supply liquid to a delivery member, such as a small tube or nozzle, for delivery to the bristles. As previously mentioned, wet bristles will readily wick up the agent fluid and disperse the agent over the bristles. It will be appreciated that a user may remove the toothbrush from the holder, wet the bristles, and then apply the sanitization or sanitization agent before brushing. This can be done instead of application of agent prior to storage, or in addition to an earlier application.

The dispenser 300 can have a small volume reservoir suitable for use by a single person for one month of ordinary use. Most dentists recommend brushing twice daily, so this corresponds to at least about 62 applications or about 31 days. As some people brush 3 times daily, a reservoir able to hold about 100 applications may also be contemplated. By restricting the volume to be about 100 applications, the reservoir is made smaller. In some cases, it may be desirable to make the reservoir a bit larger, for example, up to about 200 applications. Preferably, the pump is configured to provide in a single pump action sufficient agent for a single application to be effective for treating the bristles.

While the applicator device 300 is connected to the holder 100 in the embodiment of FIGS. 15A and 15B, it will be appreciated that the applicator 300 can be a separate device from the holder 100, with the two components being made available as a set or as a kit.

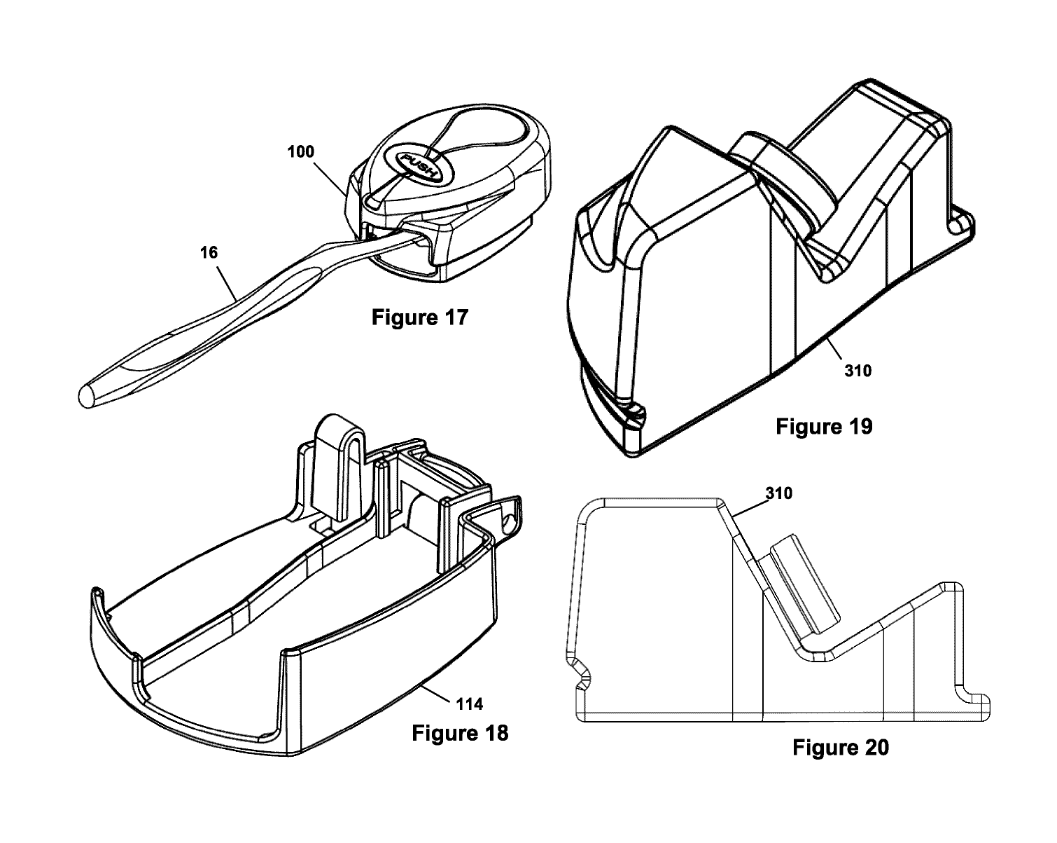

Referring to FIGS. 16 to 20, there is shown a toothbrush holder 100 according to another preferred embodiment. The holder 100 has an enclosure formed of a front cover 112 and a back cover 114 that function similarly as explained above. The holder 100 has retaining mechanism 200 for receiving the head 12 of the toothbrush 10 that functions similarly as explained above. The holder 100 includes a dispenser 300 with a reservoir 310 that is connectable, such as by snap-fit engagement, onto a portion of the back cover 114. The dispenser 300 includes a pump 312 co-operating with a pushbutton 342 that is contained within a depression 340. The dispenser 300 with pushbutton 342 functions similarly as explained above in relation to FIG. 7C.

1.-16. (canceled)

17. A toothbrush holder (100) for holding an individual toothbrush (10) having a head (12) with bristles (14) and a handle (16), the holder comprising:
a toothbrush head enclosure (110) for providing protection to said bristles (14) against contact with foreign objects, the enclosure (110) being dimensioned for receiving said head (12) of said toothbrush (10) while leaving a portion of said handle (16) of the toothbrush (10) exposed outside of said enclosure (110), wherein said enclosure (110) is provided with an opening (102) at a bottom of said enclosure (110) for passing said head (12) therethrough;
a retaining mechanism (200) contained within the enclosure for securely receiving said head (12) and allowing for release of said head (12); and
a dispenser (300) configured to be connected with said holder for dispensing sanitizing mist onto said bristles (14) of said head (12) inside said enclosure (110), said dispenser (300) comprising a pump (312) and a pushbutton (342) on said enclosure (110) for actuating said pump (312).

18. The holder as defined in claim 17, wherein said pushbutton (342) is contained within a depression (340) of said enclosure for preventing accidental actuation of said pushbutton (342).

19. The holder as defined in claim 18, wherein an underside of the pushbutton (342) has an actuating finger (344) configured to push a trigger button (320) of said pump (312).

20. The holder as defined in claim 19, wherein said dispenser (300) comprises a dispensing orifice (322) configured as a nozzle for spraying said sanitation mist onto said bristles (14).

21. The holder as defined in claim 17, wherein said dispenser (300) is removably mounted inside said enclosure (110).

22. The holder as defined in claim 21, wherein said dispenser (300) is removably mounted by snap-fit engagement onto an inner portion of a back cover (114) of said enclosure (110).

23. The holder as defined in claim 17, wherein said dispenser (300) comprises a reservoir (310) holding a quantity of said sanitization mist sufficient for at least about 62 applications to sanitize said bristles (14).
